(19) United States
(12) Patent Application Publication
(10) Pub. No.: US 2025/0195590 A1
(43) Pub. Date: Jun. 19, 2025

(54) PHAGE AND ALLICIN COMPOSITION FOR INFECTION TREATMENT

(71) Applicant: GUANGZHOU MEDICAL UNIVERSITY, Guangzhou (CN)

(72) Inventors: Ping CHEN, Guangzhou (CN); Yinglin YIN, Guangzhou (CN); Kailin YUAN, Guangzhou (CN); Baoqi CAI, Guangzhou (CN); Sifang DOU, Guangzhou (CN); Lei XU, Guangzhou (CN); Ziru ZHOU, Guangzhou (CN); Yuan FU, Guangzhou (CN); Taoxia E, Guangzhou (CN); Jiayi LIU, Guangzhou (CN); Zhihuang HE, Guangzhou (CN); Wanyu WANG, Guangzhou (CN)

(21) Appl. No.: 19/013,193

(22) Filed: Jan. 8, 2025

Related U.S. Application Data
(63) Continuation of application No. PCT/CN2024/113512, filed on Aug. 21, 2024.

(30) Foreign Application Priority Data
Dec. 14, 2023 (CN) 202311727130.0

(51) Int. Cl. A61K 35/76; A61K 31/255; A61P 31/04

(52) U.S. Cl. CPC A61K 35/76; A61K 31/255; A61P 31/04

(57) ABSTRACT

The present invention provides a composition comprising a phage and an allicin for treating Pseudomonas aeruginosa infection, where the phage is Pseudomonas aeruginosa phage vB_PaeP_GZMU_A1002, deposited in the Guangdong Microbial Culture Collection Center with a deposit number of GDMCC No:64029-B1. The phage has a synergistic anti-Pseudomonas aeruginosa effect when in combination with allicin.

### CROSS-REFERENCE TO RELATED APPLICATIONS

This application is a continuation of PCT/CN2024/113512, filed Aug. 21, 2024 and claims priority of Chinese Patent Application No. 202311727130.0, filed on Dec. 14, 2023, the entire contents of which are incorporated herein by reference.

### INCORPORATION BY REFERENCE STATEMENT

This statement, made under Rules 77 (b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: US-2024-10691-SequenceListing.xml
Creation date: 2 Jan. 2025
Byte size: 65,569

### TECHNICAL FIELD

The present disclosure belongs to the technical field of biomedicine, and mainly relates to a composition comprising a phage and an allicin for treating Pseudomonas aeruginosa infection.

### BACKGROUND

Death and morbidity from bacterial infections have declined due to the discovery and development of antibiotics, which is recognized as one of the most important advances in the history of modern medicine. However, bacteria resistance has been intensifying rapidly with the widespread use of antibiotics in clinical practice, and multi-drug-resistant strains are increasing dramatically, bringing a huge crisis to global health. One of the most common causative agents of refractory lower respiratory tract infections is Pseudomonas aeruginosa (P. aeruginosa). Despite the use of carbapenems, β-lactams, and aminoglycosides, resistance to these antibiotics in P. aeruginosa infections has become a significant challenge. However, in recent years, antibiotic resistance and the emergence of multidrug-resistant strains of P. aeruginosa have been frequently reported, making the treatment of P. aeruginosa induced lower respiratory tract infections an increasingly challenging task. Therefore, P. aeruginosa has been recognized by the WHO as a ‘critical priority’ pathogen due to its high level of antibiotic resistance.^{[1]}, and it is urgent to develop alternative therapeutic strategies to antibiotics for rational use in clinical settings.

Allicin, which has natural antibacterial activity, has attracted much attention in recent years as a way to combat bacterial resistance. It is a natural compound mainly found in garlic, which is produced by the enzyme action of a sulfide in garlic called aryl sulfide (Alliin) after being damaged or cut, and has a broad-spectrum antimicrobial property^{[2]}, with a good inhibitory effect on Gram-positive, Gram-negative, and fungal organisms. In addition, allicin has the advantages of simple structure, easy accessibility, obvious biological activity, and less susceptibility to drug resistance. The different antimicrobial mechanism from antibiotics gives allicin a natural advantage in the treatment of antibiotic-resistant pathogenic bacterial infections. An individual determination of the minimum inhibitory concentration (MIC) of allicin against P. aeruginosa is carried out and the result is 1.25 milligrams per milliliter (mg/mL).

The search for new antimicrobial agents to reduce the use of antibiotics and to alleviate the pressure caused by antibiotic abuse has become an ongoing problem in the prior art.

### SUMMARY

A phage, Pseudomonas aeruginosa phage vB_PaeP_GZMU_A1002, is identified, and when allicin is in combination with this phage, the two exhibit a synergistic anti-Pseudomonas aeruginosa effect, and there is a drastic reduction in the dosage of both substances after the combination. In addition, in facing the drug-resistant bacteria caused by the current antibiotic abuse, this combination will not only be effective in fighting infections but will also reduce the use of antibiotics to a certain extent and alleviate the pressure caused by antibiotic abuse.

The present disclosure provides a composition for the treatment of an infection caused by Pseudomonas aeruginosa, comprising: a phage and an allicin, where the phage is Pseudomonas aeruginosa vB_PaeP_GZMU_A1002, deposited in the Guangdong Microbial Culture Collection Center with a deposit number of GDMCC No:64029-B1.

The present disclosure also provides an application of the phage in combination with allicin in Pseudomonas aeruginosa inhibitor, where 1/1000 MIC allicin and 1×10² plaque forming unit per milliliter (pfu/mL) phage are used in combination in the inhibitor.

The phage provided by the present disclosure has broad-spectrum antibacterial effect and may be applied to the preparation of bactericides.

The present disclosure has the following beneficial effects.

Firstly, the allicin has no negative effect on the potency of the phage provided by the present disclosure, and the allicin exhibits a significant synergistic effect on P. aeruginosa when used in combination with the phage provided by the present disclosure, with the amount of phage decreasing by one order of magnitude and allicin decreasing by three orders of magnitude when used in combination, as compared to use them singly;

Besides, the combination of allicin and phage provided by the present disclosure effectively reduce the dosage of allicin and phage, thus avoiding the large use of antibiotics and reducing the pressure caused by antibiotic abuse; and
the phage provided by the present disclosure has a broad-spectrum antibacterial effect when used alone.

### BRIEF DESCRIPTION OF THE DRAWINGS

FIG. 1 is a graph showing the measurement results of the minimum inhibitory concentration of allicin in a specific embodiment of the present disclosure.

FIG. 2 is a graph showing the measurement results of the minimum inhibitory concentration of phage in a specific embodiment of the present disclosure.

FIG. 3 is a test result diagram of the influence of allicin on phage in a specific embodiment of the present disclosure.

FIG. 4 shows the test data of allicin used alone in the synergistic effect test in the specific embodiment of the present disclosure.

FIG. 5 shows the test data of phage used alone in the synergistic effect test in the specific embodiment of the present disclosure.

FIG. 6 shows the test data of allicin in combination with phage in the synergistic effect test in the specific embodiment of the present disclosure.

### DEPOSIT INFORMATION

The P. aeruginosa phage vB_PaeP_GZMU_A1002 is deposited in Guangdong Microbial Culture Collection Center (GDMCC for short), with the address of 5th floor, Building 59, Compound No. 100, Xianlie Middle Road, Guangzhou, Guangdong Province, China, with the postal code of 510070, the deposit number of GDMCC No:64029-B1 and the deposit date of Nov. 16, 2023.

### DETAILED DESCRIPTION OF THE EMBODIMENTS

Reagents: distilled water, LB medium (tryptone 10 grams per liter (g/L), yeast extract 5 g/L, sodium chloride 10 g/L, NaOH to adjust the pH to 7.0), agar powder, MH broth (beef extract powder 2 g/L) and soluble starch 1.5 g/L, casein acids hydrolysate 17.5 g/L, NaOH to adjust the pH to 7.4±0.2).

Biological materials: Pseudomonas aeruginosa bacterial suspension, P. aeruginosa phage vB_PaeP_GZMU_A1002 (separated from a mixture of river water and sewage collected in Guangzhou, Guangdong Province in July 2023).

Experimental equipment: bacteria incubator, biological incubator, ultra-clean bench, biosafety cabinet, spectrophotometer, autoclave, refrigerator, balance and centrifuge.

### Experimental Methods

### I. Isolation of Phage

Samples of sewage and river water are collected, centrifuged at low speed and the supernatants are filtered through a 0.22 micrometer (μm) filter to obtain the sample solution, 10 mL of sample solution+5 mL of triple concentration of nutrient broth LB+1 mL of the target host bacteria are taken and co-incubated overnight in a constant temperature shaker at 37 degrees Celsius (° C.). The co-incubated solution is then centrifuged at low speed and the supernatant is filtered through a 0.22 μm filter to obtain the lysate. 100 microliters (μL) of host bacteria mixed with 0.7% semi-solid LB agar is evenly spread on the Petri dish, and 5 μL of lysate is dripped on the semi-solid surface of the dried Petri dish, which is placed in a 37° C. constant temperature incubator for 8 hours (h) or overnight, and the phages are isolated when transparent circles are observed at the location of the dripping.

### II. Classification and Identification of Phages

The genome sequencing of the isolated P. aeruginosa phage vB_PaeP_GZMU_A1002 is carried out by Magigene (Guangdong Magigene Biotechnology Co., Ltd.), and the results are provided in the attached Sequence Listing file. According to the criteria of the International Committee on Taxonomy of Viruses (ICTV), two phages should be categorized as different species when their genome sequences are less than 95% similar^{[3]}. To assess the classification of phage A1002, an NCBI BLASTn^{[4]} is first utilized to perform homology analysis of the whole genome of A1002, and the results show that the highest sequence similarity between Pseudomonas aeruginosa phage vB_PaeP_GZMU_A1002 and P. aeruginosa phage KPP25 (Genbank No.: NC_024123) is 98.04% (96% coverage, see Table 1 for details). In order to more deeply resolve the possible classification of P. aeruginosa phage vB_PaeP_GZMU_A1002, further analysis of the phage is performed using Stretcher, Matcher, Water, and SSEARCH2SEQ from the EMBOSS suite, as well as the ClustalW algorithm from the MEGA software, to compare all known phages of the genus Kochitakasuvirus and A1002 for global double sequence comparison. It is worth noting that all the above methods yield lower sequence similarity values compared to BLASTn, which may be attributed to the fact that BLASTn mainly focuses on local sequence comparison, thus possibly overestimating the true global similarity. For example, considering the local comparison similarity between P. aeruginosa phage vB_PaeP_GZMU_A1002 and KPP25 as well as their coverage, the true global similarity is 94.11%, which exceeds the 5% difference threshold for species classification by ICTV^{[5]}. Based on the results of double sequence comparison, the genome-wide homology of P. aeruginosa phage vB_PaeP_GZMU_A1002 phage ranges from 44.9% (with Pseudomonas phage R18, using the EMBOSS Water method, see Table 1 for details) to 94.76% (with Pseudomonas phage KPP25, using the ClustalW method in MEGA, see Table 1 for details). Referring to the classification guidelines of British Association for Victorian Studies (BAVS), groups of viruses with more than 50% nucleotide sequence similarity may be categorized as belonging to the same genus^{[3]}. Given that the genome of A1002 falls mostly in the 50%-95% range of similarity to other phage in the genus Kochitakasuvirus, the P. aeruginosa phage vB_PaeP_GZMU_A1002 is confirmed to be a new species of the genus Kochitakasuvirus according to the criteria of the BAVS classification guidelines.

TABLE 1
Two-by-two comparison of the Pseudomonas phage A1002 genome with all whole
genomes of members of the genus Kochitakasuvirus using different tools.

| S/N | Name of virus | Blast | Water | Matcher | SSearch | ClustalW | Stretcher |
|---|---|---|---|---|---|---|---|
| NC_0241231_KPP25 | Pseudomonas | 94.11% | 92.90% | 93% | 88.70% | 94.76% | 56.50% |
| | phage KPP25 | | | | | | |
| OK0947071YMC17 | Pseudomonas | 92.83% | 92.70% | 92.90% | 88.50% | 94.40% | 56.50% |
| | phage | | | | | | |
| | YMC17/07/R4900a | | | | | | |
| NC_0419641_R18 | Pseudomonas | 94.29% | 44.90% | 63.10% | 89.70% | 45% | 56.60% |
| | phage R18 | | | | | | |

### III. Determination of Phage Potency

The solution of Pseudomonas aeruginosa phage vB_PaeP_GZMU_A1002 is diluted to a certain extent, and 100 μL of the diluted phage is mixed with 100 μL of the host bacteria and incubated at 37° C. for 15 min. The incubation solution is mixed with 0.7% semisolid LB agar, spread evenly on top of 1.5% solid LB agar spread on a Petri dish in advance, and cultured in a constant temperature incubator at 37° C. for 8 h or overnight. After the culture, a number of light-transmitting plaques may be seen in the Petri dish. A number of light-transmissive phage spots are visible in the petri dish after incubation, and the optimal view is selected to have 30-300 phage spots in the field of view, and the potency is calculated as potency=(10×number of phage spots)/dilution.

IV. Determination of Bacteriostasis Effect of Phage in Combination with Allicin

### 1. Reagents, Materials and Instruments

The reagents, materials and instruments used to determine the bacteriostatic effect of phage in combination with allicin are shown in Table 2.

TABLE 2
Materials, reagents and instruments needed for the experiment

| Items | Specific materials |
|---|---|
| Materials | 96-well plate, 50 mL centrifuge tube, |
| | 15 mL test tube, 2 mL EP tube, pipette gun, |
| | gun tip, tweezers, 10 mL medical syringe, |
| | 0.22 μm sterilization filter |
| Reagents | LB medium, agar, MH broth |
| Instruments | Bacterial thermostatic incubator, biological |
| | thermostatic incubation shaker, ultra-clean |
| | bench, biological safety cabinet, spectrophotometer |

### 2. Determination Method (Micro Broth Dilution Method)

The allicin is diluted to 7.5 mg/mL, 3.75 mg/mL, 0.375 mg/mL, 0.0375 mg/mL, 0.00375 mg/mL and 0.000375 mg/mL respectively, and the phage is diluted to 3×10⁴ pfu/mL, 3×10³ pfu/mL, 3×10² pfu/ml, and 3×10¹ pfu/ml respectively, and the host bacteria are diluted to 3×10⁵ cfu/mL; allicin, phage, and host bacteria are aspirated 50 μL each and added into each well of the 96-well plate, then the OD₆₀₀ value is measured, the OD₆₀₀ value is measured again after overnight shaker incubation at 37° C. and 220 rpm, and the absorbance change value is calculated to determine the inhibition effect on the bacteria.

### 3. Preliminary Experimental Results

### (1) Measurement of the Minimum Inhibitory Concentration of Allicin

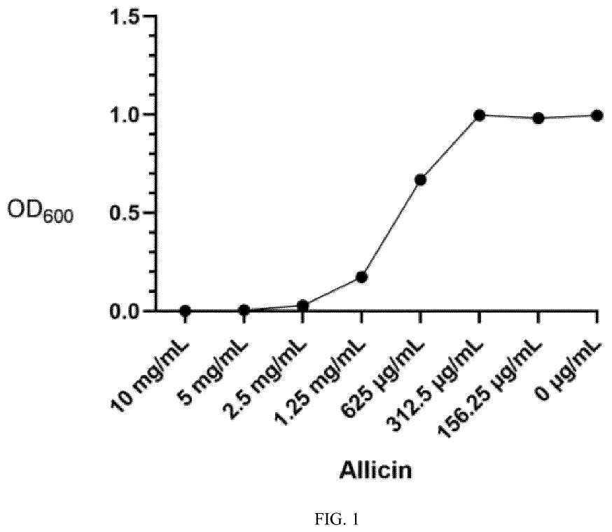

After the experiment, the test results are shown in FIG. 1, and the minimum inhibitory concentration (MIC) of allicin is 1.25 mg/mL.

### (2) Determination of the Minimum Inhibitory Concentration of Phage

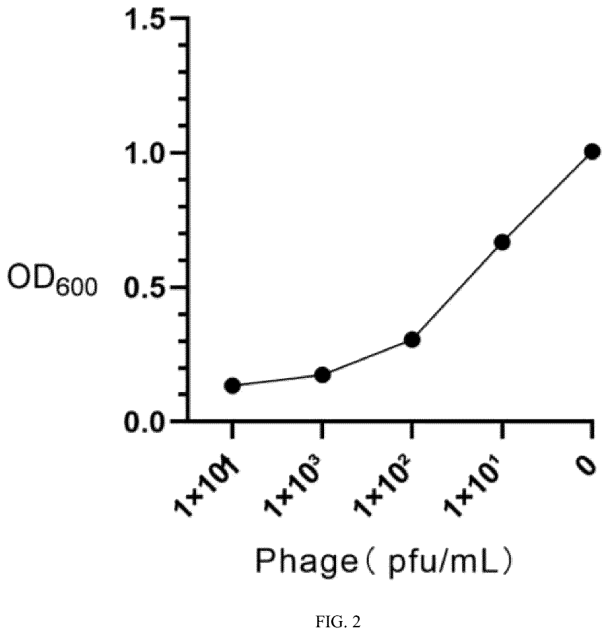

After the experiment, the experimental results are shown in FIG. 2, and the MIC of phage is 1×10³ pfu/mL.

### (3) Effect of Allicin on Phage

The Pseudomonas aeruginosa phage vB_PaeP_GZMU_A1002 is diluted to 10-8, and 100 μL of phage is mixed with 100 μL of allicin. At the same time, 100 μL of phage is mixed with 100 μL of LB as the control group, which is repeated in parallel for three times, mixed and incubated at 37° C. for 3 h, and then the potency is measured.

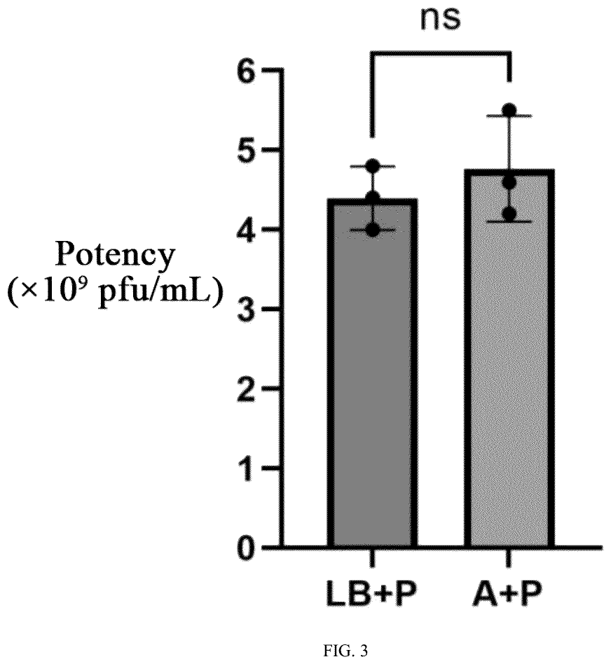

In this embodiment, paired sample T test is used to determine whether allicin has an effect on phage titer. The results are shown in FIG. 3, where LB+P indicates the control group, A+P indicates the experimental group, ns indicates no significant difference, and there is no difference between allicin not added and allicin added at 0.05 level of significance, so there is no negative effect of allicin on phage potency.

### (4) Coverage of Phage to Different Host Bacteria

The lysate is obtained with reference to the phage isolation method, and the phage is taken and spread evenly on a petri dish using 100 μL of host bacteria mixed with 0.7% semi-solid LB agar; each host bacterium is inoculated in a different petri dish, 5 μL of lysate is taken and dripped on the semi-solid surface of each dried petri dish, and the petri dishes are put into a 37° C. constant temperature incubator for 8 h or overnight, and if a transparent circle or phage plaque is observed at the location of the dripping, then the corresponding host bacterium is infested by the phage.

As shown in Table 3, the coverage rate of this phage to the host bacteria tested for drug resistance is 75.81%, and the total coverage rate is 80.00%.

TABLE 3
Drug resistance and coverage test data of Pseudomonas aeruginosa phage vB_PaeP_GZMU_A1002 to various host bacteria

| | | | | | | | | | Number of | |
|---|---|---|---|---|---|---|---|---|---|---|
| | | | | | | | | | resistance | |
| | | | | | | | | | (with | |
| | | | | | | | | | underlined | |
| | | | | | | | | | color means | |
| | | | | | | | | | more than | |
| | | | | | | Fluoro- | | | 1 kind of | |
| Deposit | Penicillins | Carbapen- | Aminogly- | Quinolones | Cephalo- | quinolones | Beta- | Aminogly- | resistance | Infest- |
| number of | Piperacillin/ | ems | cosides | Ciproflox- | sporins | Levoflox- | lactams | cosides | and phage | ation |
| strains | Tazobactam | Imipenem | Amikacin | acin | Cefepime | acin | Aztreonam | Tobramycin | infestation) | A1002 |
| P. aeruginosa′- | Interme- | Suscep- | Suscep- | Interme- | Suscep- | Resistant | Interme- | Suscep- | 1 | Infected |
| 9 | diate | tible | tible | diate | tible | | diate | tible | | |
| P. aeruginosa′- | Suscep- | Suscep- | Suscep- | Suscep- | Suscep- | Suscep- | Suscep- | Suscep- | | Infected |
| 9 | tible | tible | tible | tible | tible | tible | tible | tible | | |
| P. aeruginosa- | Suscep- | Resistant | Suscep- | Suscep- | Suscep- | Resistant | Suscep- | Suscep- | 2 | |
| 8 | tible | | tible | tible | tible | | tible | tible | | |
| P. aeruginosa′- | Suscep- | Suscep- | Suscep- | Suscep- | Suscep- | Suscep- | Interme- | Suscep- | | Infected |
| 8 | tible | tible | tible | tible | tible | tible | diate | tible | | |
| P. aeruginosa- | Suscep- | Suscep- | Suscep- | Suscep- | Suscep- | Suscep- | Interme- | Suscep- | | Infected |
| 7 | tible | tible | tible | tible | tible | tible | diate | tible | | |
| P. aeruginosa′- | Suscep- | Suscep- | Suscep- | Suscep- | Suscep- | Suscep- | Suscep- | Suscep- | | Infected |
| 7 | tible | tible | tible | tible | tible | tible | tible | tible | | |
| P. aeruginosa- | Suscep- | Suscep- | Suscep- | Suscep- | Suscep- | Suscep- | Suscep- | Suscep- | | |
| 6 | tible | tible | tible | tible | tible | tible | tible | tible | | |
| P. aeruginosa′- | Resistant | Suscep- | Suscep- | Suscep- | Suscep- | Suscep- | Interme- | Suscep- | 1 | Infected |
| 6 | | tible | tible | tible | tible | tible | diate | tible | | |
| P. aeruginosa- | Suscep- | Suscep- | Suscep- | Interme- | Suscep- | Resistant | Suscep- | Suscep- | 1 | Infected |
| 5 | tible | tible | tible | diate | tible | | tible | tible | | |
| P. aeruginosa′- | Resistant | Resistant | Resistant | Suscep- | Resistant | Interme- | Resistant | Suscep- | 5 | |
| 5 | | | | tible | | diate | | tible | | |
| P. aeruginosa- | Suscep- | Resistant | Suscep- | Suscep- | Suscep- | Interme- | Resistant | Suscep- | 2 | Infected |
| 4 | tible | | tible | tible | tible | diate | | tible | | |
| P. aeruginosa′- | Resistant | Suscep- | Suscep- | Suscep- | Interme- | Suscep- | Resistant | Suscep- | 2 | |
| 4 | | tible | tible | tible | diate | tible | | tible | | |
| P. aeruginosa- | Resistant | Suscep- | Suscep- | Suscep- | Suscep- | Suscep- | Suscep- | Suscep- | 1 | Infected |
| 32 | | tible | tible | tible | tible | tible | tible | tible | | |
| P. aeruginosa- | Interme- | Suscep- | Suscep- | Interme- | Suscep- | Resistant | Interme- | Suscep- | 1 | Infected |
| 31 | diate | tible | tible | diate | tible | | diate | tible | | |
| P. aeruginosa- | Resistant | Suscep- | Suscep- | Suscep- | Resistant | Suscep- | Resistant | Suscep- | 3 | |
| 30 | | tible | tible | tible | | tible | | tible | | |
| P. aeruginosa- | Suscep- | Suscep- | Suscep- | Suscep- | Suscep- | Suscep- | Suscep- | Suscep- | | Infected |
| 3 | tible | tible | tible | tible | tible | tible | tible | tible | | |
| P. aeruginosa′- | Resistant | Resistant | Suscep- | Suscep- | Interme- | Interme- | Resistant | Suscep- | 3 | Infected |
| 3 | | | tible | tible | diate | diate | | tible | | |
| P. aeruginosa- | Interme- | Resistant | Suscep- | Resistant | Suscep- | Resistant | Interme- | Resistant | 4 | |
| 29 | diate | | tible | | tible | | diate | | | |
| P. aeruginosa- | Suscep- | Suscep- | Suscep- | Suscep- | Interme- | Suscep- | Suscep- | Suscep- | | Infected |
| 28 | tible | tible | tible | tible | diate | tible | tible | tible | | |
| P. aeruginosa- | Suscep- | Suscep- | Suscep- | Interme- | Suscep- | Resistant | Resistant | Suscep- | 2 | Infected |
| 27 | tible | tible | tible | diate | tible | | | tible | | |
| P. aeruginosa- | Resistant | Resistant | Suscep- | Suscep- | Interme- | Interme- | Resistant | Suscep- | 3 | |
| 26 | | | tible | tible | diate | diate | | tible | | |
| P. aeruginosa- | Interme- | Suscep- | Suscep- | Resistant | Suscep- | Resistant | Resistant | Suscep- | 3 | Infected |
| 25 | diate | tible | tible | | tible | | | tible | | |
| P. aeruginosa- | Suscep- | Suscep- | Suscep- | Suscep- | Resistant | Suscep- | Suscep- | Suscep- | 1 | |
| 24 | tible | tible | tible | tible | | tible | tible | tible | | |
| P. aeruginosa- | Resistant | Suscep- | Suscep- | Suscep- | Interme- | Suscep- | Suscep- | Suscep- | I | Infected |
| 23 | | tible | tible | tible | diate | tible | tible | tible | | |
| P. aeruginosa- | Suscep- | Suscep- | Suscep- | Interme- | Suscep- | Resistant | Resistant | Suscep- | 2 | Infected |
| 22 | tible | tible | tible | diate | tible | | | tible | | |
| P. aeruginosa- | Suscep- | Suscep- | Suscep- | Suscep- | Suscep- | Suscep- | Suscep- | Suscep- | | Infected |
| 21 | tible | tible | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 21 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 20 | tible | tible | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 20 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 2 | tible | tible | tible | tible | tible | tible | tible | tible | | |
| P. aeruginosa- | Resistant | Resistant | Suscep- | Suscep- | Resistant | Interme- | Resistant | Suscep- | 4 | Infected |
| 2 | | | tible | tible | | diate | | tible | | |
| P. aeruginosa′- | Suscep- | Suscep- | Suscep- | Suscep- | Suscep- | Suscep- | Suscep- | Suscep- | | |
| 2 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 19 | tible | tible | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 19 | tible | tible | tible | tible | tible | tible | tible | tible | | |
| P. aeruginosa- | Suscep- | Suscep- | Suscep- | Suscep- | Interme- | Interme- | Interme- | Suscep- | | |
| 18 | tible | tible | tible | tible | diate | diate | diate | tible | | |
| P. aeruginosa′- | Suscep- | Suscep- | Suscep- | Suscep- | Suscep- | Suscep- | Suscep- | Suscep- | | Infected |
| 18 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 17 | tible | tible | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 17 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 16 | tible | tible | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 16 | tible | tible | tible | tible | tible | tible | tible | tible | | |
| P. aeruginosa- | Suscep- | Suscep- | Suscep- | Suscep- | Suscep- | Suscep- | Suscep- | Suscep- | | Infected |
| 15 | tible | tible | tible | tible | tible | tible | tible | tible | | |
| P. aeruginosa′- | Resistant | Suscep- | Suscep- | Suscep- | Suscep- | Suscep- | Suscep- | Suscep- | 1 | Infected |
| 15 | | tible | tible | tible | tible | tible | tible | tible | | |
| P. aeruginosa- | Resistant | Resistant | Suscep- | Interme- | Interme- | Resistant | Resistant | Suscep- | 4 | Infected |
| 14 | | | tible | diate | diate | | | tible | | |
| P. aeruginosa′- | Suscep- | Suscep- | Suscep- | Suscep- | Suscep- | Suscep- | Suscep- | Suscep- | | Infected |
| 14 | tible | tible | tible | tible | tible | tible | tible | tible | | |
| P. aeruginosa- | Interme- | Resistant | Suscep- | Suscep- | Suscep- | Interme- | Resistant | Suscep- | 2 | Infected |
| 13 | diate | | tible | tible | tible | diate | | tible | | |
| P. aeruginosa′- | Suscep- | Suscep- | Suscep- | Suscep- | Suscep- | Suscep- | Interme- | Suscep- | | Infected |
| 13 | tible | tible | tible | tible | tible | tible | diate | tible | | |
| P. aeruginosa′- | Suscep- | Suscep- | Suscep- | Suscep- | Suscep- | Suscep- | Suscep- | Suscep- | | Infected |
| 12 | tible | tible | tible | tible | tible | tible | tible | tible | | |
| P. aeruginosa- | Suscep- | Suscep- | Suscep- | Suscep- | Suscep- | Suscep- | Interme- | Suscep- | | |
| 11 | tible | tible | tible | tible | tible | tible | diate | tible | | |
| P. aeruginosa′- | Suscep- | Resistant | Suscep- | Interme- | Suscep- | Resistant | Suscep- | Suscep- | 2 | Infected |
| 11 | tible | | tible | diate | tible | | tible | tible | | |
| P. aeruginosa- | Suscep- | Suscep- | Suscep- | Suscep- | Suscep- | Interme- | Interme- | Suscep- | | Infected |
| 10 | tible | tible | tible | tible | tible | diate | diate | tible | | |
| P. aeruginosa′- | Suscep- | Suscep- | Suscep- | Suscep- | Suscep- | Suscep- | Resistant | Suscep- | 1 | |
| 10 | tible | tible | tible | tible | tible | tible | | tible | | |
| P. aeruginosa- | Suscep- | Suscep- | Suscep- | Suscep- | Suscep- | Suscep- | Suscep- | Suscep- | | |
| 1 | tible | tible | tible | tible | tible | tible | tible | tible | | |
| P. aeruginosa- | Suscep- | Resistant | Suscep- | Suscep- | Suscep- | Suscep- | Suscep- | Suscep- | 1 | Infected |
| 1 | tible | | tible | tible | tible | tible | tible | tible | | |
| P. aeruginosa′- | Suscep- | Suscep- | Suscep- | Suscep- | Suscep- | Suscep- | Suscep- | Suscep- | | Infected |
| 1 | tible | tible | tible | tible | tible | tible | tible | tible | | |
| B- P. aeruginosa- | Suscep- | Interme- | Suscep- | Resistant | Suscep- | Resistant | Suscep- | Suscep- | 2 | |
| 042 | tible | diate | tible | | tible | | tible | tible | | |
| B- P. aeruginosa- | Suscep- | Interme- | Interme- | Resistant | Suscep- | Resistant | Suscep- | Suscep- | 2 | Infected |
| 041 | tible | diate | diate | | tible | | tible | tible | | |
| B- P. aeruginosa- | Suscep- | Resistant | Suscep- | Suscep- | Suscep- | Suscep- | Suscep- | Suscep- | 1 | Infected |
| 038 | tible | | tible | tible | tible | tible | tible | tible | | |
| B- P. aeruginosa- | Resistant | Resistant | Suscep- | Resistant | Interme- | Resistant | Resistant | Suscep- | 5 | Infected |
| 037 | | | tible | | diate | | | tible | | |
| B- P. aeruginosa- | Suscep- | Suscep- | Suscep- | Resistant | Suscep- | Interme- | Suscep- | Suscep- | 1 | Infected |
| 035 | tible | tible | tible | | tible | diate | tible | tible | | |
| B- P. aeruginosa- | Suscep- | Suscep- | Suscep- | Suscep- | Suscep- | Suscep- | Suscep- | Suscep- | | Infected |
| 032 | tible | tible | tible | tible | tible | tible | tible | tible | | |
| B- P. aeruginosa- | Resistant | Resistant | Suscep- | Resistant | Resistant | Interme- | Resistant | Suscep- | 5 | |
| 029 | | | tible | | | diate | | tible | | |
| B-P. AERUGINO | Suscep- | Resistant | Suscep- | Resistant | Suscep- | Resistant | Suscep- | Suscep- | 3 | Infected |
| SA-027 | tible | | tible | | tible | | tible | tible | | |

The above coverage 47/62 = 75.81%

| | | |
|---|---|---|
| B- P. aeruginosa- | undetected | Infected |
| 026 | | |
| B- P. aeruginosa- | undetected | Infected |
| 028 | | |
| B- P. aeruginosa- | undetected | Infected |
| 030 | | |
| B- P. aeruginosa- | undetected | Infected |
| 031 | | |
| B- P. aeruginosa- | undetected | Infected |
| 033 | | |
| B- P. aeruginosa- | undetected | Infected |
| 034 | | |
| B- P. aeruginosa- | undetected | Infected |
| 036 | | |
| B- P. aeruginosa- | undetected | Infected |
| 039 | | |
| B- P. aeruginosa- | undetected | Infected |
| 040 | | |
| B- P. aeruginosa- | undetected | Infected |
| 043 | | |
| B- P. aeruginosa- | undetected | Infected |
| 044 | | |
| B- P. aeruginosa- | undetected | Infected |
| 045 | | |
| P. aeruginosa-12 | undetected | Infected |

Total coverage 60/75-80.00%

### 4. Combination Data

### (1) Allicin Alone

In the combination experiment, the bacteriostatic effect of allicin in different concentrations (i.e. allicin alone) is calculated and analyzed when the phage concentration is 0 pfu/mL.

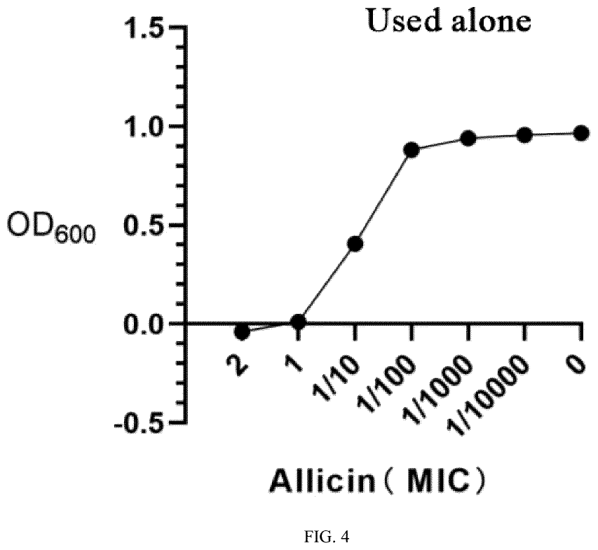

As shown in FIG. 4, the OD₆₀₀ is about 0 when the minimum concentration of allicin is 1.25 mg/mL, and the antibacterial effect is achieved. Therefore, the MIC of allicin alone is 1.25 mg/mL, which is in line with the previous experimental results.

### (2) Phage Alone

In the combination experiment, the bacteriostatic effect of phages with different concentrations (that is, phages alone) is calculated and analyzed when the concentration of allicin is 0 μg/mL.

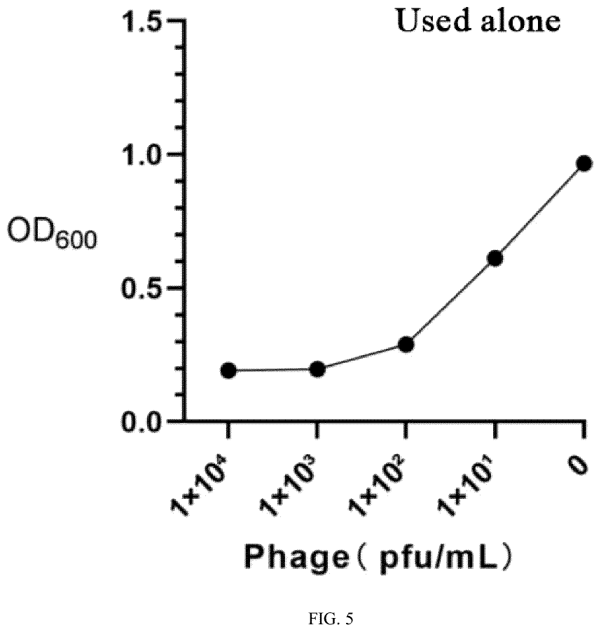

As shown in FIG. 5, when the lowest concentration of phage is 1×10³ pfu/mL, the OD₆₀₀ is about 0.2, and the antibacterial effect is achieved. Therefore, the MIC of phage used alone is 1×10³ pfu/mL, which is consistent with the previous experimental results.

### (3) Blank Control

In the combination experiment, only the MH medium and bacterial liquid are added in the blank control group.

TABLE 4
Changes of OD₆₀₀ in blank control group

| Groups | OD₆₀₀ |
|---|---|
| 1 | 0.9736 |
| 2 | 0.9601 |
| 3 | 0.9658 |

Group 1 is the parallel control 1, group 2 is the parallel control 2, and group 3 is the parallel control 3. The bacteria in the blank control group all grow, which proves that the bacteria solution is not polluted.

(4) Allicin in Combination with Phage

Different concentrations of allicin and phage are mixed in a 96-well plate by broth dilution method, and the OD₆₀₀ value is measured. After overnight shaking table culture at 37° C. and 220 rpm, the OD₆₀₀ value is measured again, and the chart is drawn.

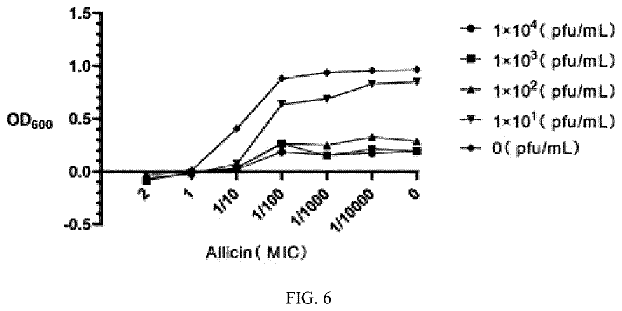

According to FIG. 6, the OD₆₀₀ at 1/1000 MIC allicin and 1×10² pfu/mL phage is 0.2-0.3, and when the hole is clear without precipitation, the optimal concentration for combination is obtained.

According to the previous experiments and the data of single use, it is known that the MIC of allicin is 1.25 mg/mL and that of phage is 1×10³ pfu/mL. Judging the combination effect according to the fractional inhibitory concentration (FIC) index^{[6]}: FIC=MIC (in combination with group A)/MIC (group A used alone)+MIC (in combination with group B)/MIC (group B used alone). In this experiment, it is known that the combination of allicin and phage FIC=(1/1000)/1+ (1×10²)/(1×10³)=0.101<0.5, suggesting that there is a synergistic effect between allicin and phage.

From FIG. 6, it may be determined that the combination dosage range that produces synergistic effect is ≥1.25 g/mL allicin in combination with ≥1×10² pfu/mL phage, and the best combination dosage is: 1.25 μg/mL allicin in combination with 1×10² pfu/mL phage.

FIC index interpretation criteria: when the FIC index is less than 0.5, the two drugs are synergistic; when the FIC index is 0.5-1, the two drugs are mutually additive; when the FIC index is greater than 1 and less than 2, the two drugs are unrelated; when the FIC index is greater than 2, the two drugs are antagonistic.

### REFERENCES

[1] Cassandra W. The drug-resistant bacteria that pose the greatest health threats [J]. Nature, 2017.543:15.
[2] Ankri, Mirelman D. Antimicrobial properties of allicin from garlic. Microbes and Infection, 1999.1 (2), 125-129.
[3] Adriaenssens E, Brister J R. How to Name and Classify Your Phage: An Informal Guide. Viruses. 2017 Apr. 3; 9 (4):70.
[4] Johnson, M.; Zaretskaya, I.; Raytselis, Y.; Merezhuk, Y.; McGinnis, S.; Madden, T. L. NCBI BLAST: A better web interface. Nucleic Acids Res. 2008, 36, 5-9.
[5] de Melo A C C, da Mata Gomes A, Melo F L, Ardisson-Araújo D M P, de Vargas A P C, Ely V L, Kitajima E W, Ribeiro B M, Wolff J L C. Characterization of a bacteriophage with broad host range against strains of Pseudomonas aeruginosa isolated from domestic animals. BMC Microbiol. 2019 Jun. 17; 19 (1):134.
[6] Patil S V, Hajare A L, Patankar M, Krishnaprasad K. In Vitro Fractional Inhibitory Concentration (FIC) Study of Cefixime and Azithromycin Fixed Dose Combination (FDC) Against Respiratory Clinical Isolates. J Clin Diagn Res. 2015 February; 9 (2):DC13-5.

What is claimed is:

1. A composition for the treatment of an infection caused by Pseudomonas aeruginosa, comprising: a phage and an allicin, wherein the phage is Pseudomonas aeruginosa vB_PaeP_GZMU_A1002, deposited in the Guangdong Microbial Culture Collection Center with a deposit number of GDMCC No:64029-B1.

2. The composition of claim 1, wherein the allicin is present at a concentration of ≥1.25 μg/mL and the phage is present at a concentration of ≥1×10² pfu/mL.


SEQUENCE LISTING (1 sequence)

SEQ ID NO: 1 - DNA, 63770 bp, synthetic construct
  source 1..63770: mol_type = other DNA
acaagttgtcgtacgtgaagatcaccactggcgagttcatcacgtaccaacctttcttcgaacgttccgcggctgaaacgaaattccgcgagatcgtggaagaccttatcctgaaacagcgaggtactttgcaatgaaaaagaactggagttcgatgggcctgaacttcatgcgttccttggctgctggcgtattctccggcagcaggttcccggccgcacgtcgcgttgactacacgccgatggcggtagacaagcagacgcctgaacaggcagaagctgccaagcgccgtgcgcaagagcgccgtgaacggcggatgaaacgtccgcaagggttcggaggctgaccatgaacatcaagcatctggcgagcatcgcccgtaccgactacacgaccatttcggtcgagttccccgagacgcgcggcaagttctacacctacaaggccgagctgccgttcccgccgaccatcagcatggtcgtggtttcgtcgccctcgaaaggctactgcttggcgcgggtcgtggaagtccacgacgtgccgcagatcgacgtgaacgcatcgtacgactacaagtggatcgtggccaaggtagacgattcgcgctacatgaccctggtcgaagaagacaaggaattcaccaagcgcatgcgggcgcacgagcagaccaagatttacaacgaactgctggcgcagttcccgcaggccatggagatcatgggcagcatcaccggtcgtgtgcacttggacaagcagcaagcggaagctctttcgggagatccgatcgaggattggtgatctatcgctgcaccataccccgatagcgcaaggctatcggggtacaccagatattgtgtcaagttcattgcatttgaccgatgggtaggggcaagcccatccttcacttccccctacccattttctcgcctggattagcctatggacatcgctttcctgtccgcacccgtgcctctgacgaaaacgtacaagcagcttcgcggacgcatcgaaaaatcgaactacccgaacacctcgtccttcacctcctacaccgaaacaccgaaacgcctcgaagatttttacgccgccatcgtcgagcacgccaagcaaggccactgcttgctcaaggggcagctcaatcgcaacctcgttgaggaatcccgcgccggctcgaccgagagcaaccaagccaccgaatgggtctgcctggatatcgatggcctggaagtcgaatccccgcaggcattcatggacgccatcggcctgggcagcgtcagttacatcgtccagtacagctcaagctatggggtgcttgacgacctgctgcgctgccacatcttctgcctgctggacaagccggtcgctgcaccgctgctcaagcaatggctgatcgacgtgaacttcaacaccgagctgctgtgcagccagatgtccctcacgccgagtggcaacggcctgcactggcccctggatatcaccacctgccagaacgataagctgctgtacatagccccgccgaacctgatcggtgtacctgatcccttgcagcctgacgagcgctaccagctcgtgaagaacaagggtacgcacttcaagctgccgaagcagatcggcaccgtcagtgcgaaccggtcgcggattgacgacaaggtgaacgagctgcgcctggaagctggtatgcccaagcgcaaggcgtcgaagtacaacagcatcggccccgtcgagttcgtagcgaacccggatcaatgcactgtcaccgggatcaaggaagaccgcggtttcgtctacctgaacctgaacggcggcgacagctggggttattaccacccggtcgacaagccggacttcatctacaacttcaagggtgagccgacctacaagaccaaggatctggtcccggactattggcacgaccagcagcgcaaggtcgaagaggccgaagagtccggcgaggttgtcacgcatttggcgttccgcgatttcaactcggcgaagtattacaacggctggtacgacccggacacacgggagctggtgctcaagcaagcagccagcgagaagcagctggctgactatatggccagtcaaggccgtgcgctgagcgatcgaattcctatctggtccatggagttcctgccgtccgaggattggacgctgaaccgtgattacgatccgccggtgatcaacacgttcgtgcagagtccttacctccaggccaagcgcccagctaaagcaccgagtgtgccgacgctgatcgcgcgtatcatccaccacgcattgggcggtgatgaagaagtaacggaccggttctacaactggctcgccgcagtgatgcagaagcgtgtagctatcggcaccagctgggtactcagtggcacgcaaggcactggcaaggggctgatctgcaacaagatcctccgcccaatcctgggcgatacctatgtccagcagaagcgcatggacgagttggacgagaagttcaacgacttcatggaacagtgcatcttcctcttcgtcgatgaagtgcagctgtccgaaatgcgtaacggccgggcgctgatgcagcgaatcaagaaccagatcaccgaagaacggatcagcatccgcggcatgcaccgcgctgggcagatgatggtgaaccacgtcaactgggtcttctgcacgaacatgcctgacccaatctacctgaccagtgacgaccgtcgcttcaacatcgcgccgttccagcccaacaagctgctgatcacctccgaagagatcgaagcgatcgaagacgagctgcaacagttctgcgactacctgatgatgcgtgaagtcgatttcgacctggcgcgcgcggttatcatgaccgagcagcgtgagcgccttatcaacaccagcagcaccagcatcgaactggcttgcgacgctgtacgccgcggtgacctccagttcttcctcgacgaactgccgagcgatagccagtctttcgtgctgcctgacaccatgctcaagttgtcccagtacaagcgcgcgcttcgtgaagtcatcgagcacgcttctacgggcggcaaaggcatagggcgggatgtgctccacaccctgctgcgctacgtgctaccggactgccccacgtccagccacaagttcacttcgctgatcaagcaccatggcctgtacgtgcagtccatgcgcatcggtacgcgcaccacgcgcggcatcgaagtggactgggtggtcccggaaggtgccctcgaccagatcgatggcgtcgccgataaaccggtcaacgacgagatttctccgaagaccaaagccgaactgaaaaagcacctgaaaggtgtagccggaggtaaagcgaaatgagcaacacctatgccacgtacgtcatgcagatgccgaccgatccgaaagcgcgtagcaaattcgtcggtaagctgaacgacctcgccgacaagttcggcgttgagttcgtagccatgtccttgcacgacgagatttcctgggaagagtacctgcgcaacgcagtcgcagaagaatcgtccatggatctggaaacccttcgtacccaatgggagaaaaatcaatgacccgtcgcactaccgcgaaacccgtcgtcccgactttcgatgtacccggctacgacaagctgcgggacgtactgcacgcagcctatcagcaggcttcggtcggtaaaggtcacgaacgccacggcagcgaagcagcgttcgaagaacaatccacgcagatcatcagcggcctgatgggcacctctgatgggatgtacttccaggccatgaagaagctcaacgagagccgcggcatggaacacgaagcgcgcaagcgtgaattgctcggcgcgatcgtgtacatcgccgggatctacatcttcgaggaagcgcacaatgccgcggaacgttcgtaatttctggatcgagctgtctgtagacggcaaggcgaccaaagtcgctaccggccctcaaggcaaagacggcggattcgacctgctggtcaagcagcgtgaccagggcggcatcattgtcgccctggcggtgataggccgcgcgaagtacgtcggtgacgagatccacctcactctgactgcggatggcgaaggtgtcagtaccgcagtcgaggaaggcggtgtcgtcgtcctcaagtgcaagaccattcgataggaggccggcatggccaagcatattcaactcaccaacaccatccgtgagcacatcgcttcgatgctcatgtgcgaagcagcaaagccgaaagcggcggacctgatcgctcgcgtgacgaagctcaacgacaagttctggcatacgcacgcagaacgtttagccgcgatgggtattacttccgacaactggcccgatctgattgtgaacggcgtatgcacttctgcgttgaccgccattccgtgctactacgaaacgaagttaggtcagtacagtagtagtcgctcggaagatacttctctcaagacgtacggagtgtatccacatatcgccttgcggactttccaagaacacggcgtactcattcgtagcctgctgagttctgatgctttcgcaccgcttaacaagttcatcactacggaatacaagttgaactacgacttcaagttgaagttcgtagcgtctgctacgatgccccgcatcgtcaacatgtccgttaaaaccgtggaggtcgcgggtattcaccaacccatgggtgagctgtgcgctgacatcaacgaagtgatccgtgcagcactcgacttccacaaacaagcactcgaacttctgcgtgcgtgccgcacttccaggcaattggaagagctgttccccgaagcgatgcggttcctgccggcaaaagcccaggagagaaaacaagacctcgcacctgtcgagctggctgccaatgtccgcagcatgctgaacaaaggcgtcccgccggtcatcaccaagtaaacaccccgccttgccaccgggaaaccggtggcttttttgttacgctgtaatgtacaatgcagcagactaatccctggagctaccctcatgcgctggtcgatcagtaagctgcacgtccgcgaaggttgtgcataccgctacaaactgaagtacatcgacaaagttcccgaacctgaacgccccctcccccctggtaagtctgagcacgcgaatgaccgcggctctcgtgtccacgaacagaacgaactgttcgttcgcggtgaagtggacaagctgccggcagaatctgctgacttccaagccttgatcgaagacctgcaagaacgcttccgcgccggcttggtcatgctggagcatgactggtgcttcgacgaagattgggaaccttgctcgcccgaagagcgggccgccatcgccatcgtcgacgtggctgtctggatcgtcaaagaccgctggctcctgatcatcgactacaagaccgggcgcaagtacgaaaccaagcacatggaccagatgcagctctatgccctggccgccttcaagaaatggccgttcctcgaacgagtaactaccgaactctggtatctcgatatcgacgagatatcgaccagtcacttcacccgaaaccatatcccggctatccaacgtgcgttccacaaccgcgtcgggaagatggaacgggacaccgaattcaaaccggccgcgaatatctacagctgccggtactgcccgtataaggacgggatttgtccgcacgcggtagacgaaaaaacgcccgtgaagggcaacgaatgggcctctgagtggaagatatgaagaacctgtacgctgaactgaagaagcgtttcgatatcgacgtagccctgtccgacacagggtttcgttttgtctacggagccatcgaccagtcggatggtgattaccgcgtaggcatggcctctttcaagatgggtctgcgcccggacccggtgatgtacaaccggtaccagtacgacatgctgaaccgcctcaaaggctacctcggtgaccatctacgcgcggaccgtgccctggccgagctgttccatgctacccgcatgcgcgcgaagctgcggtcccagcccgcagttgcgtgaaggaggactcatggcaaagttcaaaccgatcaaactgatgccgcaccagaagaagacgctgaagaagcgcggcgaaacttcgatcctgctggatctgtctgaaccgggcaccgggaagtccgtggcgcatgcaacgcatttcgtcgagcataggaaggccgggggcggagctggactgatcgtagcgccgctgtcgttgatcgatacggtatggccgcatgaaatcggtctggtcgatcctgatgtcgatatcagcgtggcattcgccagcaatcgcgccaaggcattccaaccgggccacgatctgtacgtcaccaactacaacggcctggagtacctgtccgaaaaccccagcctcatgcggaagttgggaatcgattggttcctcggcgacgaggccaccgcgttcaagaacccgcaagccaagcgaaccaagcggtgtcacaaactcgttaaaaatgtcgaattcaaggcgctgctgtccggttcgctcggcgcgaagatgatcaccgattacttcgcgccgacgctgatccttgacgacggtaaacgcctgggatggcagttcagccggtttcgcgaagcaacctgctacccggtgcaagtcggtcggcaagcgcacatgatccaatgggagccgcgcgaaggcgcagacttggccgtggctcagctcttgcaggacatcagcatccgccacctcttcgccaccacggtgaagcagagcatccgcacgatcgaattcaagctgccgccgaagctgcgcaagttgtacgagcagatggcgcgggagcaagccatgttcctcaaggacaaggtgatcaccgcggcgaacggtggagcagccgcgcagaagttgctccagatcgccagcggctcgacctacgacaaccagaaggccaacaccctgctcgacatgcagcggtacgaacttgtcgccgatctggccgaggaacgcgaaaagaccctgatcgcgttccagtggcaccaccagcgtgaagcgcttgagctgctgctgaaaaagcgtgggcggaccaccgcatatatcgatggcaccgtaccgggcgagaagcgcaaagctatcgtcgaagcgcacaagcgcggtgagatcgatgacctacttcttcaaccgcaagcggctgcacacggcctgaccctggtgaatgccaggactacgatttgggccacgccgacctacgactacgagctgttcgatcagctcaaccggcggacctatcggactggccagagccaggattgcgaagtcatcgtgatatgcgccgagaacacgctcgacgccaaagccttcgaggtctgcacgaccaaaggcaagcgcatgatgacgatgttcgagtacatccgcgacttcacgtaaggatcgttgcgatgacgattatggttccgcgcatccatgacgggatgaaacccgttacgctgcgcccgttgaagaaattcaaaccgctggtggtcgacttcgaaacctactggggtccgaagtattccctgcgcatgatgtctaccagcgattacgtccgtggggatcaattcaagattcactgtgtcggtgccaagctcggcaacgcgaagacgcgtatcttcaccgacatgaagaagttcgccgcgtggctggccaaggtccaggctgagtacgagatcgaactgatcggccacaacctgtacttcgacggcttgatcctcagcgagtatttcggattcgtgccggcggcttaccgctgcacgatgtcgatgtcgaaagggctgttccgcaacaccatcgcgcacggccttgacaccgtagcgaagctgtttggcctggccggtaaagtaaaggcccaggccaacgtcaacacgttcggcaagcgtgatctgacccgccaggagctgaccgacctgggcttgtactgcgtcgacgactgcgaagacacgtacagcatttaccaagccatgcgtccgtacataccggacgatgaaatggacatcatccacgacaccgttcgttgtttctgcgacccggtgcttcgcgtgaacggaccgatgctcgaagaagcgatcgccgaagtcgaagccaagcgtgcggtcttgtacaagcaagcgatcgaactggtgaacaccctcgctccggggctggtcaacgccaagcgcccgttcgagacggtgctgcgatctgctgtcgatttcccgctgctgcttgaagcactcggcgttgaagtgccgatgaaacagaacgacaaaggcttgtggataccggcgctggccaagaaggacacagggttcgtcgaactgtgcgaagaccctgaccctctggtccgtcttctgtgtgaagcgcgacttcagttcacgtcgaaccagaccgtcacgcgtgcgcagcgcctgctggacgctacccaggacggagcactgctgccggtgatgtacggctactgcggcgcattgtcgatgcgctggacagccggcaacaagatgaatctccaaaaccttcagcgggggtcggcgcttcgccgagccatcgaagcgccgaagggctggcacatcatgaccggggactcggcgcagatcgaagcacggattctggcgtacatagctgggcagtgggatctggtcgaagcgttccgcgtcggcgcggacgtgtactccgaattcgccatggagatttacccggatgtcgaggtaagcaaggaaaacccggatacggaaaccgagcgtcacgtaggtaagacttccatcctcggcctgggttacggcatggggttcaggaagttcatcaccacgatggcgaccagcttcatccccgtgctgatggaagtcgaagacgccaaacacgtcgtgaacacgtaccgcacgaagtacccgatgatcccggcgttgtggaagtacctcgacagcatcctggccgatatggctgctggtaacagcgggttcatcgagttccagtgcgggcttcgcctggaatacgaacctgaccggatctggatgccgaatggcctgtgcctgcattaccccgaactccgtgaagagtacgacgaggacaccgggcggaagagcggctacacctacgcgatcgtccgcgggaagaatgtcagccggaagaaaatctacggcgggctgctgactgaaaacatcgtccagtgcttggcgcggatcgtggtgggcgaccagatgcggctcatggcgaaagcctaccgcgtcgtgatgacgacccacgacgaagtgtccatgctggtcaagggcacgaaggctgcactggccaaggccacgaagttcatggtcaaaacgatgtccactccaccctggtgggcacctgaccttcccgtagcgactgaagtcaaagacgcaccgtactactgcaaataggtagatgctatgaacgtgactctgtaccaatccgaactgatgacctgcgaaaatctgaagacgggcagagtcacgtactacatgtcggtgtgcaacgccatgcggcgaatctcgaaagccgagtacgacgagcgctaccgaaaagcggacggcttgtgttgcttgcacaccgtgactgacaagcactaccgccgctcttgcaggaccgttatctacgaggttccgaaatgaagttcgacaagatcgagtacagcgtatgcagcgagtgcttgctcttcgtggcgtacggtgaaccgccggagaacggtgctgacatcgaagccgcgatgcgccgagagatcggcgaagcgaaggatgcgtacttcgtgtgcggcgtcgaaccaactgaagacgaccccgatgggatcggcgaaaatgaattcagctggcagcagtgcgaactttgccgtagcacgctcggtggtagccgctacggcgtgacgttgatgatacccctggaagaaggaaccgaggatggcagcgccgaagagccgaactaaccccaactggctggcactgcaagatccgaacaacccttccctcaacaagaattaccggagatcggtacgctactacaggcagctgttcctggcatggccgaagtggtgtgccgatcatccggggttcaagaaaatctacgacgaagccgcgcgccgtcgtagtgctggcgaggacgtgcatgtggatcacatcgtgccgatctgttccaagctggtttgtggtctgcatgttccgtggaacctacagatcataggcgcaggcccgaacctcaagaagtccaacttgtggtggccggaccatccgttcgagaaccagtgtctcttcggcgggtccgatgaacctgagcagtacgaacttcgtctgtaattgttactttgggagttgcaacatggcaacgaaaaaacctacaatctctttcgaccgcgatgccgcgatcaaggagttccacaagctatccaagcagatcactgacctgaacgcgaaagtcaaggaactgacctcgctcaaggccagcttggaagaagagctgatcgaacacctgaacatggaacaactcggcaccgtgacgttgaagaacggaatgggagtcaagctgcaagactccgtggtgccgcaagccgaggactgggacgtgttctacaaatggatgtcccggaccaagaacttccagatgctcgaacgccgcttgacggtgacggcgtaccgggagttccgcgagaagggcaaagaagtccctggcctgcgcgatttcaccaagaccaaggtcggtctgatcaacccgccgaagaagtaattcggcggggcttttcgaagctcacaacgtaagcaaagcccacaacgtaaccgagaggaaacagcctgatggctaccaaaaaaaccaccgacgccccggctaagccggccaccaagaagcctgctgccggcaaagctgtagccaccatcggcaaaggcagcaaggtgccggtcgatgccaaggcgctccaggcacagctgctggctcgtaagcagcaggtgctcgataagctgggccgtcccagcacccccaccatcgccgtgcgcaacgacaagtccttcgaactgccggacggaagtatcgtccaggcaccggagttcatccgtgccgtggtgatcgacttcgcttccgtcaactcgctgtacgaaggcccgtacgatccgaagaacccggaaccgccgatctgcttcgccgctggactcggcagcccggattccctgatcgcctcggacaagtccccgcagaaacaagcggacgactgcgccacctgcccgctgaaccagtggggcagcggcagcggcgatggcaaggcttgcaagaacacccgcaagctggccgtcgtactcatggacgagctggacgacgaaaaccgcgaaggggatcttcagttctacccgctgaacgtgtcgccgaccggtatcaagcactgggatggatacgtgcgcaatctggcaacccgcaacgatctgctgcctgtccaagtcgtcaccaagatcggcttcgacgcgcaatcgacctgggccagcctgcggttcgcgccggatgaagtcctcggcgatgaccaactggcgcaagttctcggtctgcaagaagccgttgaagacatgctgacggccgagcctgattgggctgggttcgaagcgcagcgtgctgagcgtcaagccaatcgcaagcccgccggcggccgccgcccggctgcgaaaccggctggtcgcgcccggcgctaaaccaaggaggtaacgaatgagcaccacgctggcgagtgccctgcgaacgatccgacagctgtaccgtgaactgccgacgtttaacgccgagcagatagagcgacttgtcgatctggagttaaagggcgcgaatcggcgctcggtgctcaatcgtctcgtctcccgcgcagcacagctcgctgcggacaacaaactgatccaactccaatcgaaaatcgaaagcaaaaggaaccacgagcatggcacgtcaacccgccaacaaaaccccgaaagtatcgaccctgagcagcaacccgattcccgccgacgccaagccggtaaaaggaaccgtggtagcgcgcgaaaagcccagcgttaagccggctgccgcggcggccaaggctgctcccagcaaggccgaaatcaaggccaaggaaaagcgcgtagccgagctgcgtaaactggccatccagacgcagaaggattacgacgccgaggtcaagcgcatccgcgccgaacgcgacaagaagcaggatgacgccgccaagcctgtcgcgctgctggacaaggaaatggaaaagctcagcgctgagttcaacaaggcgaaggacaagctgaccaagcagcgcaccaaggcgctcgcagcttccgagaaggtcaaggccaaggcgcgcaaggaaggtgattcggcgctggccaagctgaccaaggccaaggaagccgacgacaagaagctggcttccgacctgtccaaggccgagaaggaactcgccgccctggctaccagctaagccagcacgcaccatcaggccgcctccgggcggccttttttacctctggagaaaagcatggcgaatcccgagaaccaagcaatccagaacctgcacacgaagctgccaagcttcgtccaccgcgaaaaaatgagcaacccgtacagcaacggcacagctgatgtgtggtatgccgcgtggccgtgcgatttgtgggtcgagtacaaattcctggcaaagcccccgcaacgggcggtgacactcgacctgtcggataaccaacgtatctggcttgcggacaagtaccgcaaggggcgcgcggtcgccgtggtgttgctgttccctgcaaaattcggcggggtgatctacacgcacttgtcgtggggcgaacggaaaatccaactacctgcaccggatgccgaccacatcatcaagcgtgaactgatcgccgagttcatcgttcggagctgtacctgtgccgcctttaattcctctcttgtataccggggcgaagatcgctcggtcgctgtacctgatcgccgcattttccgcggcgacatacctcactattcggagcctacgtgatggcaaagcaaatacctacggaactgtctatcgacctggaaaccctcggaacctccgtggaatcggtgatcctttcggtggcgatggtgacgagcaccggcgaaaagttccacatagcaatcgatccgtccgagcaaatcgccgccggccgtaaagtcgatgcgagtacgatcatgtggtggctcggccccagcgctggtgaaccggcgcgcgaggcaatgaccgaactcatggccaagcggaagtacggcatggaagacgccaagctcgaaatcatgtcgttcctcgacggcgtgctgaagaaaaacccgcgcgtgcgcgtgtggggtaactccccgtcgttcgactgcgaaatgctggcgaatttcctcggctgtaaaccgtggctcttcttcaacgagcgtgatgttcgtacggcgcgcatggtggtcgggcatacccaggccagtaccggacacgacgcgctggccgacgcagaagcgcagctcgtcgacgccatgcgtttcatcgaagtggccgacaaggaggcgcgcaaatgaacatcgtggaactcattgaagcgatcggcatggacaacatcgaagcgcagtctctcaacaagtccgtgctcaaagccaataccgtcaaagacgaaaccgtggtcaagttcgtcacgaagtccatgacgccgaccgacttcgtgctcggcaccaagatcgggctgatcttgtggttcgacgcggacaagttcaacgcggccgtcgataaggtagaaagtgaaaaggccgcggaaggctaaggtccgctgccggtgccgtgaatgcggcgctcggaacaccaagcgcggcgatctgaagttcggactcgcacgcgctcggtgtcgggcgtgcggaaagatcggcacgctgcgcatcgacaaatgggccgacagaagagggtggctggtcggtaggttatgccgctgcgacggtgtaccgttcccgcaccgcccagggatggtcgggtacatcacgtactgcggcgctgagattttcgtggagtgcttacatggccaggggcatcggatcgacgaagcgccggcaggaactgtcgaagcgtgcggcccggttgaagagccagggctttaccctttctgaaatatcgactatgacgggcataccgcgcgataaggtcaacatgcgcgtgaagctcggcgagcggttactgagcctggagtgaactatgtggactttttgccctaaatgcgggtgtggagcgttcgagaagcgcgacaatctcaacgtctgtgcgaactgcgaacaggattggaatcccgaatcggattacaccgaggttgtgcaagcatacctcagcaaggtgtaccaagacaaagacgaggaaatcgcccgctacaagcagctgtatcacaaatctcttggtgcgatggaacgattgaagatcccgaccaagcgcgatgccgacgccatggcgtgcgtgctggcggtgctcgtggcggccatatccctgttggagcgcggcgggaagaaagcagtggcgtcggacaccatgttcaaccagatgctgctcgactacaaagctgctgtgactgtaggacgcaaagcactgcggcgcatgcgcggtggagacgaccaagcgtacgacagcaacgcagagcggttgaccaaggaagcgctcggcaccggcgcggagataaaccccgaagcaacgtctgtagatgatctgttcctcgacggtacgccgccgaacaagaaaggtgacctatgatcgaaagcatactcaaacaaatcgacttgaccgccaagctcggccaggacggcagctcacttcaagccatgaacgcgctgcgcgtcatccgggagactatcgccaagcatatcgcggtacatccgacccgtagcgaaggtgaacgaaagctgtcggaccttggtaacgaactgcacaacctttcgtgctctctcgaacatgagaacgaagaatgggctgaacagatcagcagtatcgcccgcgagctttgggattggccgcaagcccgcgccgccttggcgcaaccctccccgtctgccgtgcaactcagcggaagccgtgagcgtcgcatctacgtcgccggcccgatgactggctacaaggacttcaatttccccgcgttcaatgctcaggccgaggaacttcgcggactcggctaccacgtcgagaacccggcagagcacggcatcgtggatggcgcagagtggcaagattatctgcgctacgacatcggccgtctgtccacctgtgaggccattttcctgctgcctggttggtcgaagtcgaaaggcgccgcgctggaagttcacatcgccaaggcgctggggatgaccttcatccatcacgctgacgcggaacatgagcaaccctccccggcgcaagcagaagcggagcagccggaggtggttgccaagatggagtccaacggttgcggaggaaagaccctctcccagcgcttccccatcgtgaacgtcgagatcggcgaaaagctgattcgcctgtccgacttcgaacgcatcgtcggggtgctgcgggcggaaatcgcagaatggcaagaagccgccgggagatcccgttctgatgtcgtggcgtacatcgcagagcgcgcaaagttgctggaagagcgcgacgccgccctggccaaggtcgcggagcttgaatcgaaactttcggacacgcagccctacaaacaacacccgcagatcgtagggtacgcccgcaaaaaggaacttgcaccgctgctcgatccaaaccaacctgatggcagccacatctacatcgggctggaccatccggcatgctgggctgaagagccgccttacgaattcttgacccccttgtataccggtcctgtggctcagcacagcgtgccggatggctaccgtctgatccgcctcgaacacttcgccgccatcaaggcgcagctcaaccccgagcaggttgatgcgtaccgaggacggaacgtctacgacgaagacaaggtgtacgcgaactggaacgcgtgccgctcggcactcgacgagataaaggatgtgtttcagcaaatcgattgggacgccgaaaacgagctggccgagctgctcgccgccgcgcccggcaaggaggtaggtcatgagtaaggttcagcggttctggtgcgatgaggcgcggaacatccggtgcgtccgtgaagatgactttgatgcactcgaagccgaggcccaggcgctaagggaggaagtcgcagcactgcgcgcaaaggcggctgttgtgccggcggtgttcgggattgagcggtaccgcgtagaaaaaactgggaaggggttctggccatactgcgtgcgtgcaggggatggaacgcgtgaactgttcgtcggccacttgaagcaatgtcagcgggtagcggccgaattggctacggcatttgaagatgggaagtttgtcgcccaggctcagcacagcgtgccggaaggattccgtgagaagttcacgaagtgcatggaactggtgttcgctgaaatcgcctactacgttagcccggagaaagctcgtgaccctaattcgcgcgcacgcaaggcgtcggtgttggctaccgaactacacgatatgctcgccgccgcgcccggtagggaagtgccgcaggcatggctcgacgtgcaggcagagcgccgccggcaggtcgaggccgagggatggacgccggagcacgacgacctctattgcgccgccgaacttccgcgagccgcagcggcatacatcctcaacggagctaatgacgaggcgccagctatctggccgttctcggcgaagtggtggaagccgagagacgcgcgttccaactacgtgcgtgccggcgccttgatcctggccgagatcgagcgcctggatcgagcagccatgcgcagtgaagacagtgatggaggtgcagagcaatgaacctgtcttgggacttcatgaagtccatcgccgagcgggatggcgtgaaagaggtactgtcggaattcctcaaggaatcgacactcaaagtgaacggcgctgacgcactgctggttctgcacatcgtatcggcgtacaccacctgcgacggcagccacggcgggccagcatgtggtgaccctgattgcttcgccaatcagaaacccgacgacgcgaaaattcgcatgcagttggaacgcgccgtgaatgcgatgcgaacgttgtgcaacgtcgacacgaaggaagacatcgacgcgatgctgctcgctatgcgtgccatgcccggtgacttgaacgaccacatcgcggtcatagcaaccttggaactactgaaggagctgatgggatgacgaaagacgaactgatggaagcggtacgtggcttctatggcgacacgtcgcggtcgcgtgaagaaacacgcgaactgctcgaagaagtgcgtgacgagatcgatatcctgatcgagtcgctgtacgaggacgaatagccgtcctccagaaacaagaaacccggccaatggccgggttttttcttgcaccgcaaggaagcgcgatagcaaggctccatccgcaatgctcattttaccagaaacccggccatgtgtcgggtttctttttgctctgtacagagctatggggtgcggggtttcactccggcagcggatacctcgccttgatttcctcgaccttggcaatccaagcagagtaatccggctcgacgccggctttgatcgcgtcgaattcagcctcggtcttgagcgggtcactctccaagcggtaagcgtccgcccgtgccgcgaccgcagcatcgtacgcagcctgtcgacgttcttcggcctgctgctcagcagtcttcaccttactccagtcgatcattgcggcaactccaccgggccgtcggcctcgaccagcaacggttcagggaagcgagcggcgtcgctggcatcgacaggcagcgggaaacgcagggtcagctccaaccggccagagcgccgcaatacggggccttcaaaccactccgattcaatagcgtcagacggtagttcaccgccttccgggagcggcgtgaaatcgaactctacaccgttcacagtgagtacatcgccagccctgctcagtgacaggcgctcgtcgcttcccggcagcggagcaaacggtgacaacttgatgatcatcagaaccacctccccacaaccataatgctcaacccgttagtaccaaggtcttcattcaagtagaaagtaacttggtccccgcccatgtagccgcccctaacccccgccgcagccgatggccagccgctgaacggtatccagttgacgatgaggctatatgaggatcctccgataaactcagccgccaactgcactatgttgggccctgcttggtagttgctaccagaccccgtgatttggcgtatgcaaatctgagttccatctgcaaaccgaacgaactcaccattcgcattgctgccacgctggatcaccgcgccagtcggcaccccgcccgactgcgaaaccgcgccaaggatgctgtctcgcgagtacaaagccccagttgaacccagggcagctctaattgcagcactcccaagcccaagggacgtgcgcgcgccagcggcagttgcagcgcctgtgccgccattggttatgggtacggtatcgctggtagcgaactgggataggttcccgttacctccgccgttgtcgattaccttcagagggattgcgtcagccattggtcaccaccgggatattcagcgagccgccggcagcgagcgctaccggcagcatctggttggtgttcagcggcacgcgaagcgtaccaccgtcccgcatgtacaccagcacgccgagaggtacacccagcagcgtgtcgccgtctggcagctcacgaggaaagccgctgccatggacgaggggtttacgcgccgccatggttcacctcacgccagcacgatcggttgccggccgatctcgacgttgaccacggtagcgctcaccgcggtgcccaggggctgcacgatctggccagttccagtcggcggggtagctacgccggctccgggagtggtcgcgctgagccacacgttgccgggggtcatcccggtgacctgggtgttcgaaccttcgaagtacaccgtcgcgttcgctgcgctggccacggccgacaggacgaagccgtgggccatcttcgcggtgccgccggcggcgtcagccttgcggactttgaacgcgccagcatcggcccacacgttcacgtaatcacccgcggccagtgcctcggatgcttcgatgatcgcggtatcggccccgatgccagagggcagcacagagctgtccaggcggccggtcgagtcgagtgcagggatatcgccggcgttacccgcgccggccgagacgacagtcgcctcgacctcagtggggaagccgttctggttggaaaggtacttgtctgccatgtcacacctccagcgtgattgccacgccaatgtcgataaatagagtagtagggtcgagcgcgaagcctacgatcagtgagaacagcgcgccagggggaacggtctgcgtgagattgccatcctggccgaggaacacggcaccgggtgtccacgcccacccggagtgttgcacttcaccgaggcgctgcacatccaccgtgccgccaggggacgccgcattcagcgacagtcccagggtgtttatccggttgagcagcgtcgagcaatcagcgtacaccacttggttggcgtcgatcggtgcgaccatgcggtgcccggacatcggcaccgacgccaccttctggattatcgcgccgccggccgggccgggttcacccttgtcgcccttacgcccaggctgcccggccaggacgacgaacgccgccgagcgcaagtcctgcacctgctgcgcccccggctggatcagcacctcggtgacggcctgcgcgggcggcaggaccatggcggtgtactccaactgcgacgctggccgcgccaggggcaggacgatctccatgtcagggtcacgcacaagcagcgcctgcgagcgctccctggtgacaagcgcctgcggtgtcgataccgccacggacaggcactggccgacccggcggacgactagagcctgacctgcgttcatttcgctacctcggccgtgacggtcactcggccagaccccaccgggtacaccaccgacacgtcggtcgggtcgtacagttcgaggtcgtacagcgctttgttccagtccagggtggcggtcacgtccgcaccgatcagcatgctgatggtgctggtggggatatcgatcacgacgcgcccgtcagtggtgttcccggtgagcagcgcgtcagcatcgccaggggtgcggcggatggcgaaccgcgcggccagcccagtgagatccgcgggggtgtagaagacgatggtgccggctccaccgaacgccttgaagccgatgccgttcacgtcgttcagttccaggctgtccggggtcaggactttggccagatggaactgcccaggctgggtattcagctcaggaggggctttcacgctctgcacctcgaacggccagccatcagacaggccatgcccagggaccgtcagcacaaccggggcggtggccgtagcgcctgtgatgggcttgtagagcttgatcgggtcggcccagcggaaggcgaattcgaacgttttcccctggagaatctgcaaatcgatggtgcaagtcatggtgcgccctcctgtgagctgcggaaagtatgccagccggcccggagctgcgccacgctgcacgttcccgccgtaggggtagcagagtggcggttccacaactggagcgcaccacgggcctatttggtgccctacagcctgtatcgataccgttgatggtccagagcactactattaattgcaacgatgttacgaaatttccggaactggccaaaatttcgtaacacgctggagccttagtgccacggggcttacagcgatttctgttacgatgttacgattcttactactttattccactacagaaaaaataaaataaatatataaagaatatagatacttagggccgccgggggtacagggtagtatgtacgtgctccagctccgaaaagttttttcgtggcattcgtgacattcgtgacagcctagtgccacgcggcctccagctggcccaggctgtcacgatgttacgatattggccctgtccgacctcccttgttgagaacgtttctcaaaatatcgattatggttaaatgcgccagggcgatatttccgccctggaacctatcgatcgatccaatctttccacagcgcgttacccggaagagcgctcagcgcggcccccgtcacgtccccatccatcgtcttctccccggccttgagcatgtagctgaaggtcggaccgagggtgaacgccgggctttcgatgccatcggtggcgaactggcgcacgccgagcaggccggcgcgttccacacccctccagatcaggtcgcccgcgtcctggctcttgtaccagtcgggttcatcccccgcgccctggagcattgccttgaggaagtcggcggcgatcatgaacggcacgtacgcggccagggtcagcatgggcgcatagttcccattccgcagctcgtgcgtggcgcggtcgaggatggtcctctggaagctgtaggtgaactgcttgaggtgccacacgagcatccagtgcgggtcgctggcccaggctggacggtccgccgcgtgcggacgcaggactgcgccgtccacccagcggccgatggcttcgcgtaccagacggcttttctcgatggcgtccttcgggttcatgccctgggcgatgaagtcctgcgcacggaacagcaagcggccgtccgcgtcggtctgcacatccttggcggtgatgccaagttcgtcgaggtagcgtgtgctgtgctggttgacgcccgcggcgtggcgaatgatgaacttctcggcggcgacggtggccgccgagcgcatcgctgtgttccagccttccatcaggttgtacttgaacagcaggttgttggtcttgcgcgcccagtccgacatgtagttggagccgtacatgtcacccagtaggtcgagcacgaacgactcttccacggtgccgacatcctcagcgaagcgctggatgtcgagcttgttgccgttgcgccgcaggctctgcggaatgttccgtacgccggctttgaaggcggtccaggcatcgctcacttccccgccgcgtacgacgatgcccagcgggtcgataagggagctgaagatcgacagcggcagcacgacgaggttctgccaagtgatcaggccggacatggcgctgcgcaggtgcgggtcgatgttgcttccgagcgtgccttcgagcgcacggatagccggcgctacatcccgttcgatctgctcgtccgtcaggccgtaccgcttggcttcttgcagctggtccttcaggcgttcaccgttccggccgaagtgcttcgtgtactccccacgcttcgcagcctgggtgacgtagcgggtcagcacacggacgatatccgggtccatgtactgtgcgacggctgcacgatccttcagtcggatcttccgttcgttgaccgcctgcatgtacggggagtagccggccaagtcttcactcagcgccagttgcccctcggtggtggtcaacgtctcggccacggaggcggcgatgtcgggcgtcatgtcgatccagttcccgttgtcgtcacgtacctgcccgacctgcaccaacgcatcgacgaagttatcgaagtcagcgtcgatcgcctcccgactccacgcctgcgggaagtagtcctgaacgttcgccacatcgacgttggacaccttcatgtagttgaagatgcgacggagcaacgacttggtgtcgtcgtacttctgcgccatggccggatcggtcgggcgcacgttcctgtgcagcgccgcggacagatccgctagttgctgctcgtccatggtcttgatgatgttggccagctgcgtaccgaacaggttcgtcacttcccgaacggtctggatgtacccggcgcgttggccctgggtatcgctgcgcgcgtagaactggtcagcgatcttgctcaacgccgggttaccgtagttcttcaaccggccgtgcgcggtgcgcaggaacttcccggtgagcgtactcacgcggtcagccactcgactgcttaccagggcgcgctccgggccggacagggactgcatcaccacgcggttgacagcgctcggccggcgcagcttcccatccttgaatgcctggaggtacagctcggctttctgctcgttgttcaggatcgacaaggtgccgcgaatccacccagcgaaccgggacatccacgtctgcggctgcgcctggatgtcgagcatgccactggcccacaactcgtacatgtaggcgatccgctcttccgggtcgctgagctgttccaaggcggccggttcgctggccagcaaggagcgcatttttccgagcaccaccgggttactcgctgcgcggtccagcgccttgatgaactggctttgttgcttctgcccgcgaaggacggaaatcaatgcgtgcatggattcgtggaacgccgtggacatcgggctggcggcgttgaccgctaccttgatgaaccgctgcatggtcgggttcccgttgatgtccggacgggtagggtccagcttctcggtgaacataccgctgatgcccggcagcctctcgatgctgaacatgacatcgctcggcagcacgcgggtgatgtagtcgttcaggtctttggcttgctgctggctcattcggctcgcaccctgcggtcgggcactctgctgggaagtctcgctgaggtactgctggtgatacgcctccagcgcgtcgatgttgtcggacaggtacgtcaccgatcggccagccagcccagcctcgaagaatgcgtcgtcagcaatgaacgacaagcgccccaggcgcagaagacgctcagtcgctgccgggtcagcccattgcgccgacagctgcttgtcgatccctgcgccgaggatgtcccgcgcgcgctgaacaccctggcgagtgtcgaggctccggcgggcttcccggttcgccgccgggttcatggactcccggcggttgtgcaggtcttcgcgcgtggcacggtcgaaggcacgggccagatcaccagcgatctttgcctgcgccggcacggtgatgtcgagcacgggcacgccgatccggcgagccagcatgacggcgggggtactggcaccggcggtgatcaccacatccacggggtcggttccggtcggccccaagatccgcgcgtacgcgctcttgctgtacgggccggtatcctgatccacgtcgagtctggtcccgctgatggtgaacccccggcgcgtgtaggcgttcatcaggttcgtggcgtcgcggctgcgaagcacctcactacggttcaaggcgaaagcgactttcaacggcgtgccgttcatggctgcggccatcggcgcggcccaggcacccggtacgttcgtctgcgtggtgttcatccacgaccgctgctgcacactggcggcgacgttgttcatgtgcgcgccaaagtccagcggggaaggcccaggccggcggcggcctatcggcgtcggttcgttccgcgcggccgcaccttcctgcccggtggcggcttcgatggtctgcgcctgcatgcgctccgggtcgagttcctgcatgccctgctgggcggcgaagtcttcgaggttccggcgttgcagctgctcgtcggtgccggtgttgtccaccggagcaggccggcggtcgagctggcgctgccagaagtcgcgcacggacgggtcttgggcctgggcagcgccctgggccagcaggcgcaggctttcgcgcatggggccgcggtcggcaacggtcctggccagggtgtcgatggggctgccgggggtgcgcagggagcctaaatccatggccgggggcagtggttcgccggtcagggtggcggtattaccctccagagccacgccacgggggctggcagcgcgcaggagacgcgctgcggcttcgggaatggtagcggtgccccctcgccgctgaagcgctcccaggagcgtctggacggcgtattcgtcgatcaggcgctcaacgtccgcctgtcgtgcatattgcacggttcgctccgggttttcgtacgtttgtaccaagtcttggagcgcttcggcggtcgggctatccaggccgatggcatcacgaggcaggccggcgcgcggctggtctgctccctggcgcaggtccgccacggtgactgctacctcgccgctcccgtccttgtaccgggccaccacggcggtgtccggtaactggaactggtagccggcgtcggccagggtgcccaggccatcggacaaagcgtcacgcagcgcctgggcgtcacgacgaccgaatacctgcgcgcctaccagacggcgcaggtccacggctacgttcgtgccctccggcgtcgtggcagcgatgtagctgccggcggacgggttgttcggctcttggttcagccgctcaacggtagaacgacgaagctgccgtacgctgcgaagggcttgtgctgcacccgtagcgtcgggtcggactgcccgaacaaggaccgaacttgccagggcgtcaggcgacagcccaagttctgctgcaacgtctgcgggatcgacaccgagttcgcgcaaggctcgctcagcgggcacgcgctcgatagacccgtccgggttcgctcttcgagctgcttcttcggcacgctgaagtcgttgcgtatcggcacgggcgatcggggcggggataccgaggccgccagtttgaatccggcggtttccggtggtatcgccgaaccagttctcggcggcggactcgacggttcgagcttgatcgaactccccgccgtcaagttcatcgtcaagagcgccagcagattggggagcatcggatacctcgattgtgctacggctgggggactcaccgcgcaggctggcgtccaggccgtcgagcacctgctgcgggttggcgaaggcgcgttcgaggaacggttgcagctcgccggtgccggtggtgagttcgacacgcaacgcattcgacagctgacggagatcctgcggtcgcatggtgtcgcggtactcagggcgcaggctttggagcaagaactgatcgaagtcaccgcgacgttgctggtcttgccggatagcacgcgcggtggcttcattcggttccagtgcgttcaggaccgagtacgcacgctcgccgaacacgtccagcgcaccgtcgatggtgttctcgtcaatctgcccgttccgcacgcggtcagccagcacgtcaaggtactgccgaactgccggatggcttgcttcctggccctctaggatgcgatcgacctgctcgaacccggctaccggcgcgcggcggctcgcacgcacaccttcgggctgcatgcggttcaactcacgaagcgcggcgtcacgagattgcatcgtgtcggacagctgcgcgacgcgctcctgggcgatgggatcgaggtccagcacggtatcggccagttcttgccgcgtggcatcgtcgaggtccgtgcggttcagcagggactggcgttccagttcgagctgctggctacgcagcgtgtcgcgcaccaagctctgttcagcctgctggtcaaccggcaagctgggatcgatggtgtccggcgcacgtgcggcacgtgcatcgagcctggacggcatactggccaaggcggtggttgtgccgctgacggccgccaccggcccgcccccggcagcaccagcagctgcgttttcgaggaacgactgccgagcttcttcgtcggtgatcgggttcactccgatctccgggttgtagaacgactgcgcgccgcggcttaccacatcctgcgcgccttcggtcagtccttccagcgccgtcgacgtgccgaactcccgcgcaccgcggccgatagcaccagcagcgctttgcgctgcgccgccggcagcttcccgaccgagccggccgaggatgcgagcttcaggcaggatctccagaccggaactgaccacgccggcaccggtcgaggccagcgctcgttcggcgggggtagcagtggaattcgggtcggtccgcatggcgtaggaggtttcgccagcggtcggtgccaagacagccgcgccgccggccagcggaccgccgacagcggtagcgcccagggcagcggcactcaccggtgcggattcaccggcggcgttgacgacgaattcccccagggtacgggcgtcgcggatatcccccagttgctgcgccccggcctgctgcgactgcgcctcagccatcttccgatcggcgtagttgtacaggttctgcgcggtagaagacatcccagcggattcagccagggtgccggcgtaatcggcggacagcgcggcagtctgcgcgttgccccggcgcacagccgcgccggtcaagctcgtagggttgtacgggagattggggtccgcctgcaccggtgacgtttccacgtcaccaaggggccggacgggacgagtttcgtcaaagggcgtagtggccatggcggtcctgcgctaagtcggcggtgagattccgcctactatagcgcaggacgccgcttagttggacaggctgtccaaacgagtgcgcaggtccgggtccaggccagggaacgtgcggatggctgccacggcgcgttggcggtcgtccgcgctcatgttcctcgactgctggagcagccgacgggcataggcgttggccagcggagcgaattggctggttcgttcgtcctgctggcgcgaccggcccagtacgccgggattgttgctctggtactggaatccgcccaggttgtacaggttgccatcccgcgtgcgcagcaccgggtcgccgttgatgacttggatcgaatccacgttcgaatccatggtcgggtccagtacgttaccgccggccaccactgcgttcggcgtttcggacagggcgttgcctgctaccgcaccgccgaggccgccggcggctgcgcccaggatagcccgtcctgcgctgaccgcccgccctgcgggggtacggcgggcggcagcggtcaaaccgccgagtgcagcgccgccgaggccacccaggagcgtacgagggtcggactggtcctgctgcccgcgctgcaccgtctgcgctgcgtcgaagaaccgctgctgggtagtcgggtccatttcgctgaccggccgcaggctggccacggagccatcgctgttcaggcggtacacaccacggcggaagttctggtacgcctgctgtacagcggcgagtcgttcccgtgcctgcggattggtctgcgattcgctgcgcagcgcctggagctgctgcgcgtaatcggcctcctgctgctgtacccagttcggttggtactgcgacaggaacgcttgggtggcctgcggcaccacggtttcgccggactctgggtcttgacggccgaactgggcagccaaggcggtgttgaagtcctcccggttcgtgccggcgaagtttgcgtcgtcggccgatgcttgccgcccctgctgcatgcgcaggcgttcgaggcccatcagctgctggaacgcggcgttctgagcaccgagccgggcacgaagcgcacggtcaccgatctcagcctgacgctcgttgttgacgaaggtgtccgcggtgccctggcggatggtgcccatttcaccgagacgacggttctcacccatcagcgcacgggcttgcatcagatcggcggcgctaccactacggatcaagctctggatctggccttcgttgatccgcatccgctgctgcgccatccgagcatcggcaacagcctgacgacgagcgtcgcgcgcatcgagttcagcgatcgacggcacgccggcggacgagatcgtgttggccagcgggcgagcacccgcagccgaacctccagtcgtaccggatgcggcaccggccccgccggggatattggtgaacgtcgggacaccaccgacggtagtgcgctgcacggtgtcagtaccgccaccagggcgcgcgaacgacaggttggtgggcgtagccgcgccgagcggtgcgctcggttggacagcagtcgcctcagtcggcgcagcagcggcgggggagggagtagccggtgcggcaggcgcagtagcggcctcggcaacgatgtctcgctcggtcactgccggcggagtaactgccggtgccggagccgtcactgcaccggcagttaccggctcggcgacagtagtaccgcccagcgtgcggaaccgaggggtattaccctgagcgtcccgcccggtgatcgggtctaccgacatgctactttcgcctcccggtccggtcggcacgtccccgtctgcggcggacaccatgacgttctcacgcggggcttgaggtgccaaaccacgctgctggaggtcttccaacggactgacaccggaccctccaatagctcggtcaagggaacgaacagcgccatcaccggcggcgtaccccgcactgcccagggcagtcgctccagcaataccggtggcaagggcgccaggaccagaaggagcgccacccacaccgccagcagccgcgccaagagcacccccaacaccggcacccaggagtggggcggcatcgaagccaacctggcgagcagtctgtccaagatcaccttcggttccctcgtatacgcggcgagcattgtcgatagccggaatagcaccagccaatgcaccgactgcgccgcccaaacgtcggggggctacgcgggcctgctgcgcgccgccgagcacgcggttggacttggccagcgcttcctgcacaccggacggcatgggttccggcggaagcccacgcatcggcgcggtaatggtacgcgtcgcggcgttctgccggatacgcacagccgagtcgtcagcagcgcttgcggtgggcggcagcccgctttcaccgcggcgcaggggccggcgcgttccgtcgccaccggtgtagtcgatctcgtcgacgggaatctcttcgacgccgtatccgagcggttgacgagccatgcttcagtcctcagttaaaattcttcgcagtattctatgtcggtccagaatttacccacaaccggcggctccggcgggagcagcggcacgcggtaaatctggccattgtcgaacagcacacacaaaccatcctgcactttccccatgagcggcgaaagaataccggagctggggaatccagtgtattcctgggtgaaagtcgcaccgataacagtcgacgaaaggatgccgttgttcccgaggacgtaaatcaggtcgtccagtcctttgacgaatgcaaccggttgttggtttgtaggtacgggcgaagtctgctgccaagttccgccgatgcgagcgaagtacgagtcgaagaaattcgagccgtcattggtaatacgcgctgatacgatacctacttcaccatcgaagtacgccgcgggtgccgggttcggttgctgcgcgttgacagcgatctcgtaagtgctcgtgtcgatacccaggtaaatctgcccgtcgttaggatacgccggtttgcagataaccagactatcgtagaaatacgtggtggccggtacaataggatactcaacatgcgtgagtatgctgctaccaacaataccgccggtaaggaggttaccgccattgctggagttccaagcattgcgcccagcccaactcagcgcaccctctttgttgttatagaacgtctggatagtgaagccttcttggttaccgggtagcatctggatcagttctagattgaccagatcctgcgtacggtagatcgctctgttccacgggctggattgcatttctaccacaaaccagtagtaccccatggtgatatcgtagccggcagtgacgaagtttctataaccgctcggcatcgcaacttcggtccacgacataccagcgtcatcggagcagaacaggtaactcacgtactgcccaccgggcgtgtacccggagctggtaagcgtaagatgtccgtcagtaagcgacccgtcaatttgggccaagtcgccagggttgatcgctgggggcagccccgtagagttccaagtcaaaccggcgtcgttcgagtacaagacgttcaaagaaaattgcgtacgaccgacgagaaccaacatgcgttggttcccatcggtgaccaagttcgagacgaaaccaactccgtcgtatacgagttcgggagtgacagccattacgggttacctccaccggccgtagcgctggcgatctcggcctggagcgcagactgggcgtttgcggcagcagcggcggcgtcaccgaacgcgcgcacagcagtggccagggcatccgccgagttgttcacgttggacgcagcgatcacgttccgatcgttgctgatgccgccttccatgcgagactcagcggcgcgcagcctgatctgctcgaacccaagctcgacttggtagtagttggccagcgccgaggtcaacgagctgtaggccatggctttgattcgcccggcctccaagtccttgttcgggatgtcgagccactggcgatagaagtcggccagtgcctgcatgatgccgagcttcagccgaatggcctgctcttccgcgaacttcataagatcgagtttgatctcttcggcacggatggtttgcgcgcggttcacgtcagcgatggcatcggacatgcgctcttcttcacgcgatacagcaccgaccagggcaccgggagggagggagaatcctcggttggagaaatcgacacggatctgctggacagcgctttcacgtgcgcgagtctccctgtcgcggctgatgtgccattcagcttcgaacaccgacttgtccaggccgaacggcttctgtccggtaaggatgccacacagccactcttcgggcttggtgcgcaggcaggccgtcaactccgggaaatactgctcaagccacttctctacctcgccgtcgaggaacgccaccgtctcaccagtcgtatcaccgggcagcaggtcaccgaagttcggcggcggctcaatgaccggtttctgtagcgtgtagctcagcgacggcttcgaaagcggcggcgcgaagttgttctggatacgggaggtggtttcccgcgcagtgcgcagggcatcgttaaccgtgtcctgcaaaaagtcgatcgtatcgtcgatggccatgttcaaatcccctggatgtctttgatgcgaggcgcgatcatcgtcccgcagtaccgagcaccagacgccgagccgtagttggcgatcttatcccaagtcgcccccgagtcgtagctgatgtaggtgaagccgtgttcaggtccaggctccccggcgaattgtgctttcccgccgatggagaacagcaccgtagcgtcaatcagtacctcttcagtgttctcgtcgacgacttgcttcactggcgcgtccatcagcccagtgtagtaccagtcacgcacgccgttcttcaattcctcttcgactacgccggccagctccaaggtgttcttgttcacgtcgtacacagccatcgccatgttcccgtcgatgaaaccgatgtcctcgttttcctggaattccgaagtcaccgggaacaggaatttccccgcgcctatgtgcaggatgttcccgtacatggagttacggaaccagttgcacgggcggaacgggaccatctggctgaacggaatactcacaccgtccttggtcgccaccacgatgtcgatactcacgttcggtccgatgttcccgttcgggtcatggatgaagtcgctactgaaaaacacgcacacggcattcgtgccgtccgaatcagctccgtagatgtactgcatgtcgtgcggtacaccagagaacgtatcgatagttcccttggtcggcccggccaccgtttcccttcgaacgatggtgccgaactggttcacccaggcgcagtctacggaccaatacggcgtactgatcccgccattcggatcgctcggcggatcggtctggttgatgctgaaggaatcgaacagcacgctgtcacctgtgtcgagcagcgccaagccggacgggaaatgcgagttcgccatgtagcgttcggaatccccgttgatgatcttccacggacggcggtactgatcagcttcctgcctggaatcgataacgctgctaccgacgagcagcgccgtgccatcctgctggatctgcaagctggcgatccacaagccgcgcgcgccatatcgatcttcatcgtcaaccatgtcgaagacagcgtgcgccaccagcagtacacggtagaacaagccgatgtcttcgtcgttcccatgcccgatcgtgaggccgcgggaccacggatactgcgcgttgttgaacgtggtggccgccgggctggtagggtcatacggcggtgggttcacgcgtttgctgaacggcatgacgttgttcgcatcgagcatgatcgagtcagagacacgcatggaccacggctgtgtcttcggcgtatcctcgccggggtagttgccgtccgactcgatccacccagttcccgtttcggcgttggcggtgtacgtggcacgcatgaacgacggcgcaagtacaggcatgtcgtagaagaacgtgtctgcgcgcgacacgaacgcgaacgggttcacgtcaaagacctgcggcatgttcaggtagttttcgaggtgaatgatcccgtagtcaggcaacgcgaacgcgctcggtcggaccaggctgccggtggcgacgctctgcgagaacgtccccagctccatcactctgtagccgcgcgtccccatgtggctaggtagcagctcctgcaccacgatttgctccggcggttgcttcaagggcttgaggaacgaatcccttccgagcgggaggcggatcagcggatggtgagtctgcgcgcgggacgtgccgttgtacgggttaccgcacaccacgacgtaggcttcggtgtcgagcaccatagcagtgccgagcatgttgctgcgcacgccgcgaacaatatacccgtcgatgatctcgaccttggtggcgtacttcatggggaggttgccgagagactgggccagtttctcggcgcgcttcacgaggtaggtgccggaaatcgtaaccggcttctttgcgatggtcaggcgcatcagttcctccgatggattctgcgggaggaaaccccgacctccacttcgatgttgtccagatccgcgaacgaagcgtcttcgacttccaggcgaagcctccagtggcgagcagcgacgcctttcgcaacaccgtccggcgtgcggaactcaggactttcgccggcaagggtgtacacgcgctcttcaccgtcatcgccggtgatgcggaagtacacacgcccgtccgtggtgataccagcgtacgcgctcgacaaccgcttggtgttcttcacgccgtagtcgtcagcaccgaagtcgatcaacgcgcggagcagttcaccatcgtccgtctcaccttcgaggcgatacaggccgtcgttcttgatcccgaacgtgtcattacccacgcgggcaaagccggtgaagccgaaattctggtaggtggtcagcgcgccggtcagcacgttgacggcgtactggagcgcagtctcccgcgcggtgctgatgttcgagtagaccgccagcccgtcgcgcatcatcagttcgatggtgctttccagagtgaacgtcgtgttcaggccgaggtagtcgtaggcatccaggctgaccagcatgatcaagtcgatggtgccgctgatctccatgccgtccgtgaccatgagcagcacgggtgcgtcgagcatggcagtgctgtatacaggcatcggctccggcaccatcagggtgcccggcggccagaggttttcccaaacgtcgatgatgtaccgagacggccacttgccaccggcttctgcgtacggacgatccgagatcaaccgctgcggcgtcgggatcgtgttgtccaggccaagcacttcgccggtgaggatggtggtactcccgaacggccgagggatatgcgccgacacgtaggaccagggagctacgaaatcggacgtaccggcctggagtaccaatcgcggcaaagtccgcaccagggtcgcaccggagtaattcgaaatgttccccaacggcatgggaacgatgccgttgatgacctgcggctcgacagcatccgcgaccaataccatccgcggaagttcggaatcgacgccggcgtaggtcgcttcgtagatggtcgggcgcaggcgcgggaccagtgcattgagctgcgcagacgagttcccaccgaacgatgggtcgttcacgaagtcgccgaccgagtacagcgtggcgtcagcgtagatcgtcccgccggaaggcgtgatgccattgacgatggggcgaagcgaagaatcatcgttcgggttgacttcgtagaacgtgacccggccgccgacgcgcgacatggcgtaccggttggtgaaccggaagaagtcttcgaagatgatcacaccacgatcgtagatacggaaccggtcagggtagaaggcgaacgcatgtgtcgggtggccgaacgagttgtcgaagcgtccatcagagaacccaacaaggacgccggtcggtttatccggcacggtgaacttcaggatcgcatcaccttcaacagccgtcacgctgcggccgccagcgttccatccggtcttcggatcgacgatggtcttcccagggataccgggttcggacggtacggcaggaacgcagatagtcacgtcgacgactttccacatcgacgtgtactgcggcaccttgtcggtgtactgcggcacccctaccgtgtacgaaccggagttggtagcgctattggcgttatgcgacccgaagtaatcgtcgccgatcggctgaccgccgtttacgttcgggacaatacgccgctcgacatagcagtacgccgggcggccaggaacggggggccgctcagggaacgtattcgtttgcccagtttttcgcagcccgttggccattacgactccggcatggtcaggtagaagtattccagcgactgaaccgcaccgctggtgaggttaggctgagtgatgttcatgtccgcgccgctcacagcaacggtgccctggatgcgcggatcgatgttgctcgccgatccatcgtccgtgggctgtaccatgcggaagaacgacgcagtgccagtcgcaacacaggtgccgttccagacctcggacagcgatttggtcaaagtgccgttgctgacggtgctttcgaagctcacgccgttgccggagccgttcacgctgatctctaccaacagcacggccgagccgagcgtagcatcagcggtggccggagccgagccggagtagattcggattaccgagccggtcagcaggttgcgaagaggttggtcggcgatcatcgaattgcgcagaccagagctgtacttcagtgccatggttgtgtcctcacacgatgttggtcggcacgtacgtgatgccggagataagttgggcggtctgcccggtggtgagcggtttcggggatgcgaatcgtactaccgacagaagcagcccgttgttaccgcctttggtgctcgacgacacgagaaacgcaccgtacacggtgcggtcctgcgttacggtgaactgcgcgcggttggcgatgttgtccatggtgcctactccgtcgtacgctcggttccacacggggcgagtcgtttcggaatagttcgtgaactcctgcatgttggttgggatgtcagcggcagtcgtcgaagacgacgggatgtagttgccggcgaacagaccgcaatagaacgtgctgatcggcgcggtatcgccgaacggcgaccgaataaggaaatccacaccgacctgcgggatcaggttgtggacgatctcttcatccacgacggtgccgtccttgtcacagatcacgagccggtgaatgaatccgtcgaggatcactgcgctactggtcattgtcgattacctcaagctgccagaagtctccggctccgagtgaattggtggtcgtttggcctttcagggtagtgacgaccaactggttaccgttgtgttctagcacgccggcagcaccatcagtcgcaatgtcgggagcatagtttacccggtttggcagttcaatgttcccactggcgtcgcctatggcttgaccgtaccgggtaaaccaagccgcgcggccatcgggcagcttgacagcggtcccgcgcactgcgccgaagttcgtcttgcgcaccgaactcggctcgtccgtttcgacatcggtcagccagtaggtttcctcgtcggtggcgacgaacacgccggtgtcgacgctggcgagcatggtgatgtcgcgcggtagctggatgaatcgagtgatggggttggtcaagtgcggccacatcggctcggtcatcacgatgatgttgttgtaggccccaacgatgacagcgtggtgcgacacaaggagcgagcacggcggcataggggtcatttcgtcggtagtgaaccgctgagtgttatccaccgtggacgtgatgatgaacgtgttcagactgaccagcccctgggcgtacaacgtcgatccgttcacagagctgcaatacaccgtgcaggaccgcgggtcgttgcacgaaatctggatcgctttaccacccggtacggacacggcaacggcgtcagcaccggactcttcaccctgctcgccataagctgtcacagccaccttgtacgttccggtcggcaagtggccgtcgacgagactgatggtgtataccggcgacggcactccccactcccgaaccgtgttgccgtcgaaacgaaggctttcggtgggggtagacaggtacagctggttgttgtgttcgcagccggccacttcaccgttggggttaaccgacccgacgacatgcccctggttggtcctggcgtcgaatactttgaggtcgccgccatcgacgaacagaatggccgtgccgagcgggaacacggcccggccagcggcgcagtcattcaccttgacgaagccggtacgcagctcgaatttcccaccgttggtgggatccatgttcaccgccttgacgacggagttctccggcagccgctccggcttggcgatgttgttggacccgctggcccagttgtcgcgcgtctcgatgctcattgcgccagtaccttcttggccttctgccaacggttttccctgtcctcagctccattcatcccgccgttgatccggcgggtgatctcgctgaaccgaccctggtccgccagggtattgcagcccttgtctcgccacgtagcggcggcggcttcgaggccgccgtcgtaggtggccatcatgtccgggtcatctacgaacgggcgtccggtcatgacctgtgccctgcggtagccatccttgccggtgacctgcaacgggccgcgaccacgataccgccatccatcaccactgagcacgtcgccattgcccatgcggttggcgtacgtgttgttggcgatggcctgcgggttgcgcgcgatttgcgtagccagggcattgggaccgccgctgtgtgctttgtagcgactgggccacacagcggctaaacgggcagcgctgtaattgaggttctcttcgaagcgggtgaggccggaggattcatggccgatttgagcgaggtacgccgcagcacgcgccgacgacgtgatgtcgtagcggaccatgctgcgtaccaggggttcgaatacttcgtcgggagcgtcggggaacaacgcctcgaactgctgcttactgatgaccatctaagacctccttcagccatttctggagttccacaagacggtcgcgggaatccttggcgattaggttgttgctggtcgccgtttggagagcttccttgtcatctgccgccaccatcagctcgaactcggccggcactaccatcagacgctggtcgggtttcgggaacggggcgcactgctgcgtcgaatccaagggaggcgttgtagacgcgcacccaatcaggaggaagaacgcacttaccagcagcggggttttggacatagcggacgacctctttaacgacggtttgagtccgcacctcggtgtctttttgttgaacatccacacggtcggtgctggccgccgtgattttagcgtcttcgcgcgctttttgcacagctgccgcccgctgccggtcagcagcttcgcgggcctgggccagcatggcggctttctcggcttcgcaacggtccccgcggatcgtccacccggctgcgccgccgaggccggcagcaaccagaaggctgagcaggatcaggaatggggttggttgcataccctcaccttgttgccgatgaaggtgttcaccaaggaccacatgaacatcgccaccagcatgttgaagccgatcaggatcgaaagcccgtacccggactgcaccagccgggcaggctgctcggaccaaatcacgaccatggccaggatcgccagaaggctcatggcaccgataccggccacgagggtcttatcatcttctcggcacttctcagggagtcggaaaaactcgactacgcgggcgaacgtgatggcccaaatgagcgacgaaatgatggtgatggggagcatcggagcctcctacgggaaaacccgtttgaagaatgccagcacccgctctacgacggtagcttcgagcgccgtcaggatcgggtaggcaaagaaaccgagcagcatcagcaagctgtctcggtactcgctgggaggtacgaatccgccgagcgccttcgcggcgaacatggcgatgaacacgttccccaggaacaaagagacaacgaacttcttgcctcggttcaggaacacatggctagccgcgccgccgaacccaccaagagccatggcatatagccaatcgctgatgtccgaccagaacctttccatgtctacccctaatactcgttgtagcgaatcggctggaacgagcggtggcttatggcctgcgactctttgatgtcgccgagttccatgccgaacagccgaagttgctcattcgattccgacttgtcgaacacctcagcgtcctgtacgagcaagccgaggtacttcatgtagaacagcacggcacggtcgtaccgagaattgatctccagcttgtcgttgatcgtcaggccgcataccgggcgacggtacgcagcgatagtgagctggagcgcggtgttgggcttggggcggtagagcgccagttcgacatcgccttgctgatcgaactgcaccgtcaacgtacggttgctgccgtggcagttccaagcatcttgcacgccgaatgccgggagctgatcttctacgcagttgatctcgacttcgccgcgctgggattcgatccaggcgcggcgagggcggatgatcgcctcgtccataatgacgatgccggtcccaggctccacctcaagggtaaactggccgaccagacagagggttttctcggcgatcttcagctgagcttcccggatgtagctgaccagcagatcgttcgaccagaagtaaggctggaccttatcccgcatttcttcccggaacgtcgtgacaagatcagctacacgggtgcccatggccttactccggcttgtcggtctggtacagcttccaggcggagtcgcgttcagcggctttcacttcgaagccggtgatggaggcgattgcttcggtcttcggcttaccgccggaggcgaagtcggcagagtcgccgcgatccaccagagtcttgatggcggccaggatctggtcgacgcgttcagccgcgttgttgtcgatctgctggtcgacggtggtagcggtgcccggctcgccggtgagcttcagggaagcgcgcagcttctcgtcggtgatgctgttcacatcgacacaaccagcttcacgtacagccttcagcacgaggttgtggttggggatggaacgcggcacgccagcttcgaaccgaaacgcgtgaccggtgatggtggccacgacctgatcggtcagggagacgtattggacggtgttctggacgggggttttgccagccatagtgatgtgctcctacgctgttcgttgggggatgaaaacaggggagtctttcgactccccaagccccgtgaagtgtatcagttgtacgccggggagccgtaagtctcgttgccgcgaccttcgacgatgtaggccagcaggaacttgaacttaccggcggtgccaccagtggaagcggtagcagtgaaggtgatcactgcgccgttcgggtaggtcagcttggccggagccaggactttcgcgccggtggttttcagatccagatcggcggcgatgccgtccgggtcggctgccagccccggctcgatgacgccgatgtccagggttgcagcaccggcagcgttcgatgcggtggtgaccagcagcttgctgatctccgggacgaggatcgcgccgttttgcagttcgagggtcagggtagcggcctgaccgtcgaccagatcgctgaaatcgagggtctgctcgatgaacagcgggtactggcgtgcggtgttttccagagttgccattttcctatcctccagaggactggggaacgtggccgcttaggcggccacgtacacgttgatgacaccgaagtcctcggtgttaccgtcctcgtagatcgacgggaactgaggcttcaggaagccgaggatcttcgccacggagataccgtgcttgttgtcgtagtcgaattccttctcgacccagcgcgggttgccgatatcggcgaagcccagcgcctgcgcgccgcagaagagcatcgagcagccgtcaacgttgccggaggcaccccacttggcacccggagccatgccgcgggtgttgtacacgtggcggaactcgtgaatcaccagaccgtccacggtcacgatgccgcccttgaacagcgggttcacgttgccgcgggtgtaaccgctacgcaggttggcgatgtagtccgggtccagcttcagcttggccatggccagcgggtcgaggaacacgtggtacatttcctcgccgccgtcgccgcgcagaccacggatgtacttggtcttcgcgtacgccttgaggttgaccagcagagcgtagctcggggtatcggcggcggtaacgtcggaggtggcggcgttcggaaccagcaccttgttggtgccatcccagcgcagacgacggttggccgaaggcggagtaacgtcggcggcgaaggtgaggttcgggaacggcgagctaccacgcagcgcaccgttggtgcgataggcatagctcacgccagccagcgacaggaacgccagttggtccatacggtcggccagccagtacgccagcatgtcgcggctggtggtacggaagttgacgatggaacgctggtcggccatgcggccttcgtggacgttggcggcgcgcatctggtccagctggatgacggtatcgaaggcggtcagcgcctcttcgttaccttccagttggttgtcgccgaccacaccgtcgccaaccatgtcagcgatcagggtgattacggcgcgtgcgcctttctcgtcacgacgcagttcggtgatctgctggatcatcgagttggcatcggtgccgaggaacatgttgatgaacgcggcgttacgcgcttgccgccagaactgaagggaccatacggtcttctgttcgttggtcaggcgcgcaaagttggtgtagttaccttggacgggcaacggcatggctgcatcctcttctgcggcatagtggaaagtggttcagttctgaggtgtaagctgcgcgcttaccggcggacttgcggggaggtgttgcgtggtcgctgcgcggacctggcgaatgcgcaaactataggcacaaaaaagcccggttgcaacaaccgggctttgtgttccacgtggaacatcactccgcgtaatcgaagtcgccgcgtgcttttgcctgctcagcttcggacatcgactcgatatctttcagcgtttgcggagtacgcacgagcgcggaagccgcggcaggcggtgcaacggccggcgatttcggctggttcgacacggcggcggttgcgcgggatacggcttctttccgacgttcctggcgcgcgcgatcctgcggcgtttgctgacccagcggtttcaccgcagcggcttgttccacgcccaggacgtactcgacggcacgggtcatggcgatgaccttgccgtacttcggcgacagggactggaacagctcgttgatctcgtcgacagtgccctggtcgaacgcatcggcgttgttgtcatccaacacggcgtgagtagaccgcagattggtcagcagctcttcgaacttcaggttctccagctgttcgcgggcgcgctcgtcagcgatgctctcggcgcgcttggtagccatcgcttcctgaatcgcgcgcatgtccttacgaatctgcggcagcagatcgaagtcgttggccttcatcgcttcgatctcgcgcgactctagcttgtcgtagctggcctggagatcgaccagttcagaggtcagcgcgcgggatcggttctgctgctccagcaggtgctcggcagcccggcggcgctcagcctccttctgcatgcgggagcgcggaaccatgttcgggatcggtcggcccttgtcgtcgagcttctgctccggctccggttccggttccggttcctgctccgtcgcggacttgtcctcggtcttgccctcggacttgtcctcggtcttgccctcgggcttgtcctcggtcttgccctcgggcttgtctttggctttagcctcggccgagttcggctccgcgggattgtcgccgcgatcctccgggaactcttcttctacggtcttccgtgcaaagcgaccttgttcatcgcgcgccggagcgtccgctttcggtgcagggctgtcgtcgaacaaggcagcgtcgccgcgatccccaccctcgttgtgcgcacccggagtggcgtcgccggcagggatggaatcgatgtcgatatcagcctcggcaccgaaggcggcagcaccttcgtcgatcggggcggacagtacgtgtcgaaggaaattacgcatcagtggattctccgttcttcgcgggggtaggtttcggctcttgggcggccttctctgcttcgatcctcattttaagccgttccgtaaccgcgtccagtcgcagtttctcccggtcgagtgccagctcggcagttttgatcttgagatcgttgtctaccttggtctgttcgagcgccaaccgcgcggcttcgatgtccacacgctgctgctcgatgtcggcacgctgctgctcggtatcggtgccggccttgagcgcatccgtctcggcctggacgttcttgacgttggcctgggcggacttgttcgcggcgtcggactgcgcacgctgcgcctcggcggtgactttcgccagattcgcctgggcagtagccagttgcagctggcgcagctgctgcgtctccggcgaattcgcctcgctctgcatctgctcgatgatgtcggctttgtccaacaggcggctgttctggataagcacggtgtcggggatgttgatcccttccttgcgcatcgccagcgcctgatcgaactggctgtcctccagggtttcccgcgcaggcacggactggacggtgacgaggtattcacccagggtcaggtcgttggcgatctcaccttccggggtcacctcgttgatggtgaacgactcgtctttgttcacgccgccgccgttgatgtacagcagtcgctcttcgtagtagtaggtctgcactatgctcaagatcgcacgggccaggaacacgtcactgcggtcgaggttgtcgaggatacgagccatgccggcgcgcccagccatgcggttggtttccagagcgcgggcagacacgtcctcccgtgcctcaccgcgcatgtagtcggtcacgccggagatcgccttgatccatgcctgggccttctggttcatgccttccaagccagccgggagctggttcggctggatcttgtcgatgtcgttcacgtcctcaacttcgagcaccagacctgtctcggcaccgcgtgcctggaggtcgtcgacggtcatgttcttgagcgacccgtgcttcaccttccagccgctattggccgtggtgttgaggatgtgcagctcctggctggcggttttgttcaacagctcctgcacgccgaacagctgctcgacgataccggtggtgtgcccgaagcggaaatgcgggaagtacggaacgaccgtcaggtactcgtagggactccatccgtcgtgtagcaaggtagagccggctaccacggtccaacggattcgcttggcgatctgcgaaatctgcacgatggacgggttccgcatcaggtactcagcggtcttcaactcgtcccagctgtccggcacaggacgcagctccccggtgcgcgtatcgacgaacagctcccggcgggtcagtacgcggtactgccggtcaaccacgcggatgtaggcatcctggcgctgggtgccctcttcggtcttgttgatgtactccggcaacgcgatccgatcccggtattggtccgggatgtcattggtctgcgcagaacggtactggtcggccagcgcgcgcagctcctgggtcttctttttcccgtacagggcttcgaggctattcggggacatccagcgggagacgataacgtcttcccaggtcgcagggtcgtagtcagtcgcgtcggggtcgatcagcacctctttcgggttcagggtcgtgacggccacctccccgtacaggttctccgtgaagtccaggcgcacgtcgtagaacccgcgaccggtgatgttcccgtcctcgaacacgtccgtacgcttccacgtcaggtggttcccctggctgatgtgcttgaacgtcttgttcagggcggtggcggttcggtcgtcgacgttcccgtaccgcgggctgaagcggatctcgttccgcccgccgatctgctccccgaccatcacgtccatggtctggagcaccaagttgatcgtcagggccgggcggcgctgcgcccgcaggcgggtgatatcgtcctctttccactggtcaccccaccagaagttttggcacttctcggcggtgagaatgaacttgtcgtggccgctgtcgcgcatgtagtcgtatcggcaccacgtcgcgttgaccagtaaggactcgtcgttcttcgacaacgtgcccggtagcggcaggtcaaaggtgtatgcggagttgatcatcgggcgttccccatcatgcgctcatgtgactgccgttcccgacgccgcgcaggatgtcggacaactgctgcgcaacggtgcgatccccgcgacgcggccggcgcgcggaaagagggagcggtgggttcttagtaaggatcagctgtgccatccatgcaagcgcgtcgactatatcatcgtgccggcctaccgggaatcgaaggaactcagccttcatcgtctcgatccagcgagcgccgcggcgcagcgtgaccatgtggttcgccatacggccctggagcggccgggcacgtgccgatttatcggtcagcggcttcagcagttcgaccttgacgtagattttccgccgcttggcctcggcttcgaagtacggcctgatcgacttccaaatctgcccgtcttccacacccaggacagggttgtgcgacttgtaccgctgcacggtgtcgagtatggccgtgaccaccttcgagctgtcccccgtgcgcatacggatgatgtcgtcgacgtggatcgtgtcgttcggcatgtgcaacccggcggcgatgacggtgtagtcactggtctgcttctcggtgatcgcgaagtccacggtgatgtacgccgtgccgtcgtagcgctcgtccccagggtcgtgctcgatgaagtagtccgacttgaagtattccccgtcgtccggcaccgggttctgctggtacaggctgttccagtcccgcgcgtgcaagatccgtttcaggttgagcaggtacttccggctgtaccgggcttcgtgcagagcttccccggccttcctgaacggtacggcgccgggcagcggcgagtcgacgatcgtatcgtcctgagccagccactcgtcgttctcggccagggctgcgaagttgacgatcttccaacgctcgtattcctcgtcgtcctgcatggcggcgcagagccggccggacaggtcgtcgtcgtgccagcgggtgttgatcagcagcacgccgccgccgggggtcaaacgggtcagcgccgtcgaggtgtaccagtcccaggtcttctcccggatcgtctccgaatccgccgactcccggtccttgaacgggtcgtcgatgatcagcatgtgacagcccataccggtcacgccgacgccaacgcccgcggcacgtacaccgccgcctttcgacgtggcccagtcatcgatacgctcgttgaccagttccaagtccgggaagatcgccttgtattcctggctcttcaccgtgctgaggatgtcgcgggacatcttctcggccaagtccatgccgtagctggccaggatgatccctgcctccgggtatttccccagcgcccaggccgggaacttccggctggtctgctcggatttcccgtgccgcggcggaaacgtgaccattacgcggggcgagcgcccattcattgagtcttcgagaaattgctccagtacgcgcgccaggatctcgtgggcgacggctacctcgtacttcgagttcatcttctgcacgaacgggaggaaccgccggcgcatgagcgtgcgctcggccatgatcttctgcgcggcggccagctggtactcgtggctatcccggctgacgaccccgatactgccgtcctcgtagatcacgtgggccgaattcgaccgcgtgttctcgcgcatgatcttcgggttggcgcggggcttcttcttgtcgatccggtctttcttcagcttggccgttttcgggatcggcacggtatcgacgggatcattcgctacatcgacgatcttgttccctcggccggcgacgcggcaggacagacagatcccggtgctgtgcctgggggtatagatcggcgattcgcacttcgagcagtgccagcgcggcttacgctcactcgacgtattcggcatcttcggctccttcgatttcctcggccgacaagatccggctgttctcttggatcaccttcaacaactcggcgtcggtcttggtctgcatctgcgctgcgatctgcgctgcgccgccggtgatctcgatcttgaccttggcctcgtacataccgtgcatgagaccgagttcccggtagccggcgatcatcgtcctggggtcggccatctgccgggcgatggcgatggcgtccctgaagccgtggatgatctcttcgcgggtgacctcgctgatctccatgttcttctcaaacagggtgcgtagcgcgcgctgcacgcccacgcggttcatcaacttcttggcgtctgccgcaggggtagggaagttcgcgccggcggcggcatcgacaggactctgcccctgggccacacggatgcagaactcccgctccatcgggtcgagatccatcagcgacttcgcgcgggtgcgctggagcttgtagtgccgctgctcatccggatcgacgaacgaatgctccgacttgacctcagccactgcggtcgatgcgcgtgacatggcggtggccgccttggttttcgagggcggccgccggggagtggccttggtagtggtcgtcttggtagcggtcgtcttggtagcggtcttggtgaccttggtagcggtcttggtggtgcgtgccatgtcgtactccacggggatgggacggctgtttcatggcacaaagtgtagcggggcagggagtaggtggtccatccatggaccggtcggcatggctcgcaggttcagtctacgcagctgccgaggggcgcgtaaagaggtcgacgccgatcagatttcgagcgattcactgaccctggtaagcgccagctctacatagcgcaggcggtcgttcagggagtcaagcgcgttgaacaggggactgtgtacggcgggagacatcggtatttcgttgtctaccttgacggtagtcatcctgaccggtgcgagccgagctgccagctcgtccactgcacgatcgaggcgattgatagtgcgctcgatctcgatgacggcatcggccacgggcgaagaagtagcctgcgtggcttgtgcggcctgtgcggtgctggcggatgcgtagccgacctgtccgctacgggtagagggggcatcgaacggcgatacgggggtaggtgcagacatggtagtcactcctgtacggggctggtgaggccgttgggttccaggcgtacatccatccaggcgcggaagccggccagttcccacagcttgtcgcgggcctgatccaaggctttgccggtggcgatgcgctcgccgatctcacgattgaagttgttcgggtccacgcaggcgctgatggcagtagccagggtgaactcaccgtcgagcagcgcgacggcaacggtcgtggtggtgccttctacgacgtggcagcggacgaccacacggtcgaacaggctctggatatacgacgaggggatgcgcggggcggtcaaacccttggctttcatttcctgctcggtcttgctctcggattcgatggacattcgatcactcctggggcttgttgagttcgtggtcgtcggtggtgacgagatcggcgttcgcaccggtgatcgctcccgacagtgcagacggttcagcgtgcatcagctcgtgttcagcacgttcgatctcggcgcgcttggactcgatcacgtccttgttgctgtaggaaccgtgcttgccttcttccacttcacgagccagctcttcgatggagggcaggccgaaggcttcacgagcgcgttggatgatgccgccgtacgagttgaccagatccatgtgcagggtggcgagttcgcgcattgcgtcgtcggcctggaactgatcttcgtcgtcgatgcactgctggaggtagctgacccgactcttcagatactcgatatcgttgaggtgggtaacgcaggctccgatggtgacttgtttcagggaagggttcatgccgtgatcctttgcggttgggttgaacgcgatgaatgtagcactgaaacgttcaacgtgacaaccttatgggttggtggttttattttgaaactgctctgatggcctcgcggttttgttttagagggccatgggagcgtgaatttcgcggttttgttttagatggccatgggagcgtgggggcaggtccacccctccctctcaaaaaccccactccccacttcggattcgttttcgtctccggccaaaagaattcttaccccctccccctatgcctctcgtgattcgcggagcgaatcacccttactccttgtgtctttcatccacactggagttcgcatcatgaccttgctcgctcgctacaagaccttcgtttcggttctcaaccgtgtgcttgctctcaagctcagcctcggcttgatcatcttctgcatcacctacgtcggcatcagtgcctttggcttctatgccttgtggttcacctacggctggtggtgcgtacccatcctcgctggcgctaccgtggtcgtctactacacgcagcgcttcttcatcgacgccatcttcatctgacctaccgtgcgcgctgcggagcagcgcgctctctgtctctacgcattcattcacttcgaggatctaccatgtctactttcgctgctctgaagaacctgggttcggccatcgtctccgacatcgctgctggcgcacgcgcgaccgcggctgcaaccggctcggcctacgagacggtcagtgccaaggtcgcgcacaagcgtggcgagcgccagatcatcaacgagttccgcaacatgctgaacgagcatcctggcgagctgctggcagcgatgcgcagcattcaggatcaactcgctcagccggccaagcctgcacgcaagcctcgcgctcgcaaggcggctgcacaatgagccagtcgcaggtcaacgagttcctggctctgctcaaggtcaaccgtgagctgatgtcgcagctggccggcacgttcggcctcaccatcgaaatggctgaccacaacctgcgtactgcaatcgagcacatgactcggatcaacgccgagctgctgctcgcactgcaccgcatcgaaaatccatcgcaggtctgacgacgagggcgcaccgggaggtgcgccctttttgtcattgtggcaattccgccactttatataggactctgtaccatgacctacatcaccgatctgctcgattccaagtccccggctgcactggctgctctgcgttccaccgcgaacagctgcctgatgcgtgccgtcaaccaggcactgaagctggcgcgcatgcaactgcgtcatgtcggtcgcggcatcgacggtgccaacgacctgcgcgccatggctgacgcagcgaagactcgcgctcagcagctggacagcgccggccgggacagcatcacctccgaggaagtcatcctgcgcaacttccgcctgtactacgccatcgccactcggctggaggatcgcgacagtgagtacgagcgcgcgatgaccgtcgaccaggcattcgagttcgtgaccactccgcgtgacagcagcaagctgtcgcagtccgccgagctggctgcgctgtcgaagatttccggcatgtcgcctgaagacatcctgaagatgcgcgacctcgacgctcgccggtacgcggactcggccaaggatgcgcagccgctggcgcgtgtcatcctcgacgacatcccgcacaacggtgtcagcgaggacgagctgttcctcgaacttccggtcgagcagcagttccagaccttcgccaaggccaccgaggcagcggtggtgcagctgcaacgcgacacgcagcgtgcctctcgcttcctcgaaagcccggtcaaggcactgcgtgacatcgcgttcagcgacgtgatggtgagcagggccgatgcggactccctggagaaacagctggagcattacatcgtcactcacaagtccgagttggacttcgtgtgatacaccggcgctcggctcttcggagctgagcgccatttcctcgttccaccaaccctcaacagcgcctgggcgctatggacaaaatcatggacatcatcaaaagcagcaccaacgaaatcgataccgacgccgtgatggaagaactccacaacatcatggccagctacgccgagcgcggcatattcctgtcgctctacttcaccgccaacatcgagatcaccaacaaggacggcaacgagtacgacttccgtgctgcacgcggcatggtcagcgtgccgggctgggtacacggcggcggtatcggcgctgtcctgggcgctatgggtgaaggtgtcgagttcaccgacaacccgatctccctggtgctgcgtgctgctcagcacgccgcggcgcaccaacaacttatcaccgagcacatgaatcaggtcggcgagatcggcctgatccagcttgtggataactccaagttggaggtggtgaaagccgactgatttccgcacgtgtgtgaggggatcggagggggccgacccgcccccttctcccctccgccctttctgtatcgatcgctaacctaagtggttgtttcttgaaaccaattagctttttctatcgctgtaacgctttcgttccaaacaggatgagactgactctcaacaaccgtcacgatgttacgaaattccgtccaccgacgaaaatttcgtaacacgctccagcccaggcagcactaggggcacagagctttctgttacgatgttacgatcttttcttctttattccactacagaaaataaaaaaataataatagatatagttaataagtatatagggccacatatacactgtatctgtagcggaatagcgcagaacttcgtaacattcgtgacaaaagtggctcaaacccgcgtggcactaaggctccagcgtgttacgaaattttttgagatcgtgacaatcgtgacaaaatttcccggccaaaaccccgcaaacccgcgtggcactaaggctccagcctgttacgaaattttttgaagatcgtgacaaaacctcggtgccacccggcgcaaacccgcgtggcactaaggctccagcgtgtcacgaaattttttatgtactttccgtcactccgtgaccagatccttcatttcagtcatccacaggagcctggaaatgcgcgtccgcgacgttctggtattcgctttttgcgtcctcttcgccttcgccaccatgacggtcttcgtctatgcccccgcaaaggccaggaaagcgcatttcgaggcgtgttcggcccaaggtggggtgttggtatacacccggtacgaaacccctctatgcgtgaaatctgacgccttcctgtccatccctaactattaggagatcaccccatgttcgtcgacgctctggccttcggtttcatggctgccatcggcatcttcatcctgctatggcgtctgcctcgcatcctcgtcctgcatctgctgggtatgggtttcctgctcgacacggcgttcaccaccctggtgttctggatgcactggggcacggccatcggcatgttcgtcgccacgatcggcggcctgttcggatcggcaggcatcagtctggccaaaggcgtgcttggctcgatcgacttcaagaccaagcagtacaaagctggttggttcggtgaccgtcgcccgcccgagtacaagaccactccgccggccaactgagcacccgctcacccgctcacccgcccacccgcttacccgctcaccctccccacgaccaaaccctcattgccaggagcacgccatgccgagctacgccgactaccccatgcctaccaagcacaaacgcttgttcaccaaagtcgaggtatcgaaagcattcgaagccgatctggacgaccgcctcccggaagtgagtgccttggccatgaccagaacctacgcagactatggactgcgtggcacgtccagctatgactcgtacgtgagcggtttcatcttcgacaaatccaacgctcccgacgccgttccgtatatcgactcgaagactcgacttgccaaagccaggacgcagcgtagtggctcgttcgacaagcatgtattcagcctggctttgattcgtcgcaaccccacccccggcacccccatgaagccttccaggaaccaactcgatcggctcatgtgctacagccggttcaccgattgcttcttcgatgactgctcctttcagaacattcacatagtcgatgcaacgttcacgaactgtgttttcttcgacggttcgttcgtcagatgcgtcggttaccgtaccaagttcatcaactgtattttcctcggtacgtgtttccacgaaggcacgtgggcgaacgccaaattcgacggctgtaaaatgtacgaagtacgtttccgcctatcggtagatcctttccccatgccggagatacagctcaactacgtttccttctacatgtcgaaactcgtaggcgtgaccttcgaaggcgacgtgttctacgacaccgtttttccagggtgctacctgatcggcgtagctctgcaagatgccgatctgacgaacgtggatatgcgcaccgcgttccacgtcgatatcgaaagcatgcgcggatgcaggctcagccgaccgaatccgacgcagttcatgtcgctgaacatccacgactacccgatcatctacacctcgaacgtcatacagatcggttgtcaggcgttcagcatggaccagctgcgttactgcatgagcatggacacgcaggagctgtatcagctgtcttccgatgcgccggaattggccaaagcccatctgaagtggctgctcaagaccatcgagcgcttcaaacccaagccgctcccgtacgagtgttgaacgatgaaccctcccaagcgttatcgccccgatgcggggttcgagcgccgtgcatggcgtccccacaactacctgctggaccacccgcggttggaatggaacccgtacaccaagcagcgcccgtgcagggacttcgattcgtcccagctggtacagctccaactgttcgtcgtgggtcactcacgcgaaatcatcaaccgcatcatgtgcgctgcacaggagtaacaccgtgctgatcgtccacactcacctcgaccctcggcacatgaaccctgaacccatcacgccgccggcaccgttgccctacgtcagccggcgggcgctggcacgtgtccgtgagtgggttccgccgccggatgtgtgcccgaactgcggcagtgaagtgaagctggtcaacaacatcgagatttaccgcagccgatcgtacggggaatggccctacgcgtaccgttgcgtgaacgatcgctgtgacagcttcgttggtctgcacaagcacaccgacctgccgctgggcacgttggccaccaaatccatgcgtgacgcacggaaggactcgaaagcagtcttccaccagctgatggaccaatggagcggcgtcttcagtcgctcggaaatgtatacctggctcgccaagcagctgggtatcccgaaagcgcactgccacttcggaatgttcgacatcccgacgtgctcccgtgcgctgaaaatctgcctcgcagaattgaaggaacgtagccacttcgaaaaatacccaaccttttgagaaacccaaccatgtccgacatcaacacccacaccgagatcaccgaggtcatcgagaacgccgagactacttcgcggacgcagccgcagccggactgcatcaccatcctcccggatgacattgccgcgaaaacacccggcgctgtctcgatgtccttctaccgactgctgtccatccgcgctacgttgcgtctccaggccgccggcataaagacgcgcggcggatccatccgcaaacgcatcgcggccgaattcggcctcaagcccagggacagctacgaagcgttcatcgatcgggcgaacgtgttgatcgaagaagccaagctgaaagccggacaacgcgctggtgtgctgcccaaggattgactggagaacgtaatgctcgacaagaaacgcgaccgaatcctcgccaaccagaccccgcatgcgcggaaggtctacgaatgcgtaccgatagctgaaccctggccggcgcatcagatcatggcgcagttgaaagtcgatcgtggttctgccttgagcgtcgcggtggttcgtggctgcttgaaggctctgcacgaagctgggctgatccgccggccgaccagcgatctgtacatccgtgagaaggtgactgaatcggcggccgccacgatcgccaagccaacccagccaacccagccagcccagccagcccagccagcccagccagcccagccgaaggaacccagcatgtccacctcgatcgacaccctgctgaaaatcgcacagcagctttctgtcctggccgccgaagtcgaagagaagctgtcggagatcgcctccgagatcgagacggcggccaagcaggtcgagtccaccaccgcggccgatcgtgagcagctggccaagctcaagcagctccagtctctactgaaggatctgtgaccatgatcaagaccagaaaagagccgcgcagggatctcgaagagatccgccgcaagcacgaagagtacatcgccgagcacaaggccagcggcggtgtgacccaggtctaccagtgcccgttctgcgcccagcagatcgagactgcatcaccgtctaccaagcacaacatgtgggacaccatgacgagttgcccctggtgcgacggtctgcatttcaagcgcgtgtatttcacgcacgtcgaggtaagagtatgagcacgaacgaacttcccaacccttccatcggcgagaagatgcgcctgcggcattactacggctacatgcgcctgcgcttcaacgccgacggcaccgtggacgggcagaaggggtcgaactgggggctgctgctcaccagccgggagtggatgaagacgctgaccttgtggcgtcaagggaggtaagcatgatcaccacgatctaccacgtgaccgtcgatgtacccactctggtctggaatcatttccgcgacgaccggatttatttctgtgagatatgcgaggcgttcaaaggaaacgtagggtctggcaccaaccactacgatgtggttgaagaatgggccgacttccacgattcctccaccgccgtcgccgctgaggcaaaagtcaaagcgctgatggtgaagtacgaacgcctcgccaaggtacttgaggaacaagctgaagaggaagacaagggtgaccactaaacctgtccctcagaagatcaccatcgaaggtgatatcgccacgaacgtacgcaagcgcggatatcggctcgtgcgccgcttcggttccataccgactcccggcgacgaaggcttgtcatccaccgccatgtggccggtgctgctctggcaacacgctgtctacccggatcggttcgctgtgtcgtacggcgctgaactgacctattgcctggactgggtacaggctgccgaacgtctcggtgctgccctgctccacgccacaactgtcgacgggaaaatcaacgatgactagcagcagacaactcaagttcggttcgtacatttccggcaccatgcgctcgcaggatcttgtgccgaaattcttcgagatcctgatgtcggtatcgacgaacgccgcgtaccagatcgtgctgactaaggcattcgaggacgacatttcactgctcgaccattggctcttcgaaactatcacagggcgcaaagaatggccgcatgccgaccacggttggtggctctctgtcgaatccgtggaaatggtcgatatgctgatcaacgcgcttcaggacttcgtgccggtaggtcattacttcggcgcgcaccctggcgacggttcggacttcggttgctggcgcttcgaggactcgcatctgttcaccgtgttcgtacgccagagtaacaacgaaggcaccacgtacgtcgagtcggtgtggacgagtaacgccaccgaagccgccgagataatcctcgccaactgcgccaatgagtgggagtgcctcgcatcggaactgcgcgtgttgggcatcatcgccagcgagacagaaatcgaaatcgttgaatggaacgacgaggaatagaccgtggacgacatccaagttcggctagccaagtacaaagaccgtaaagaagaatctcgatacttcaaaaccctcttctataactacggcctcagcttcggacatccgaatggcggacgtactcccgaccgcggtgagttcacgacctgggtcgaaccctcgcttcgattcgtaggtaacgcggatagcttggcgcgcatcgggcaccgaggctggtacatcgatctgtcccagcgcgatgttgcatatgggcaggtctatcgactctcggccaggaatggtgaagaacgcttcctggctgcctacgtctacagctacgacactgactgcgtgacgttcgacatcactgaggtgttcgacgacaaacgagaagcagccctggccgccgacgaaatggccaggatcgccgccgagcatagtcgcgaagctgatcgggaagacgagatcaaccagcagaaagaagaggcgcgcgaactacgcgctgaggtacgcacgcgcatgcgtgcggtgatcgccgggctgcgttggaagcgcctcgcagccaggatgatgccaccgacctacatcccggacatcttcaacgtgctggccatggaagaattgagccgcggattcaaagcacgcgctgagctgtacaagctctggaagaacggacccgataacctataacccgcaacatcacactacgaggaacaccgtaatgtacgacttcatcatcaccctgaacatgcacagcttgtgtcgtcaactttcggcagcttgcagggaagaaaagggatacgcatcgcaactgatgaaccttcttcgcatcgacaaactgagcgccctcgacaacatgtacgaagcaatgaatttccgcatgactgccgagcagttcggccagttcatctaccttcgtgcggccgccggcgctaacaacgggatcaaagaactccgaatggaacgcatcctgtccggctctacctacaggttcaacgtgttccgcgatttgacggaaagcgttccgaacgaatcgccctcggcttcgtcgactgcgccggcacagatggacaatgcgatatctgtggctgtcgatatcggcatggccgccaccgggagcgcatcggcgtctaccggcggtatgagccactacgcagcggagatcgatacgttgcgtgccctggcctcgaaggtcgaagacgcacagatcgtatcgatctgcggcggcatcgaagtggatcgcatcagcgcactggatctggtgtacggcgaaattcacaagcgtgaacaggctatcgccagcatgttcacttccaaccgtcaacagtaaggatcaccaccatgggtagtcacgttgcaatggcgcattacctcgaccccatcacgggcgaaaaacacaacgagcacattctgctcgataggcaactgaaagacaggctgccggaccatccggtagctatcggcttcgaactgaacgacgagcacaagaagctgcatgccgacggctacatcgccctggtcgctatcgaccgcgcgaaatccctcggcaccagaccgcaggatgtgtatcgcctgggcatccacatgcacgttcgccgtgaagcactcaagcacatcctgaacatcaacgacgcggatctggcactgccgctgatttgggtcgatgtggaagtcatcgacaccatcaaggcaatggccggtatcgaagaaggggatgccagtgcagaagaaaccgagtaaggacgcagcattgaatgtgctggagttcatcgccaacgaagccctcgcagcgatcgccaagttcgaagcagtgcagagcgacgacaacatgtcggaactcttcgctacccggacacgcttgaaggttcagctcgacgagctgagggcagcacgcggcgaatcactcaccaacgatctgtacctcaacgggtcgttgcgtgacgtgaagtcgccggagctgctgatgctcaaacgcggcgaaatcgagtccatcgagtacatcaagcgccgggcgacgatcctgcgcaagaagctcggtatctcccactacaacgcactggagttcctggcgcgcgaagctggtttcgattcgtaccgccatgcactccgagaactggagtatggcaagtgaccaccccgttcaatgaaggttttgtcgttatcgaacacgaacccgacgagacgaaaggcgaactcgcggagtacctgtatgctcccgcgacgtacgacttcgctctgcccgtagagtgggtcaagcgtgccgaagccatcaccggtcagcaggtagtaccgcatttcgtgtggttgtacctgccgaacgatgtagctggcatgcctgcgccactcaccctcaccggcgtgcatatcctgatgcagttagcgcgctacccgtaaaccgttcacccactcgaccactcaaccattcatttctctacacaaggaacatcggatatgtcgaacaacgccaccgtcaagaaagccgtcaagtcgctcagcgagatccccgatcagcaccatgtcgccgagatcgtccaccacggcgacaagctgaccctgcccgaaggcatgggcgtcagcgatgccatcgatctgctggagcgccgccgcagctacctcgaagaagctgtcggcctgacgcgcaccttcaacgtcttcccctgggacggcgcgaacgccatcaacatcgccctggcacgtcgtttcggctgggccgccgccgaggctacccctggcttcttcggcggcactccgccgaagatgatcaccatcgaagtcggccctggccagacccgtcgtgtaccgtggggtcgcttcagcatgccgggtatcgatggctacgtggcgaccagctccagcatgaaagacggccgtctgtgcttccagctggttgcggaggttcggcgtaaggacgagcagaccatcgagaagctgttcgatgaagtcgaagacgtgctgaagcgcgaatcgatctacgccggcaaggcgattaagatccgcttccgtgacgacgacggcaacaccctggaaatgcccgagccgaagttcctcgacgtgagcggcgtgtcgcgcgacatgctggtgtactcgaaagaagtcaccgactccatcgaaaccaacctgttcacgcccatcgagcgcgtcctcgactgcatcaagaacggtatcccggtcaagcgcggcgtcctgctgggcggtccgtacggcaccggtaagaccctggccgccaccgttgcatcgcgcctcgctgtcgacgccggcgtaacctacctgtacgtgccgcgtgcggacgaactggctgacgccatcgagttcgccaagcagtaccaagacgtggcctgcgtgatcttctgcgaagacatcgatcgctgcatgagcggcgagcgttcggtggcgatggacgacatcctgaacatcctcgacggcatcgacaccaagtcgtcgcggatcatcaccgtcctcaccaccaaccacctgaccgacatcaacccggccatgctacgccccggccgcctggacgcgatcatcgacgtggtaccgccggatgcggaagctgtcgttcgcctgatcaagctctacggcgctggtgacatcgacgagcaggctgatctgaccccggcggccgagctgctggccggtgccatcccggcggtcatcgccgaggtggtcaagcgctccaagctggcccagctccagctgcaagcgccgggcaccgcgatcgagaagatcaccggcgaagcgatcctcctggcggcgcaagccatccagagccaggtcaagctgctggccgagcagtccgcaccgcgcgtggcaccgctcaccgtcgacggcctgttgcgcaacatcatcaaggacgagtgcgaatcgatcagtgacgagctggccgacatccgctccctgaccgacgctatccgcgaacgcgtgtaaacgctcaccacgttagaagcggctccttcgggggccgcttttctataggagttcatcatgacgctagctttcgatttttcggggtttttgtatttcgcttctggtctgattttcggcgtgttaatcggtccggcttttttctcgttgttcaacgccccgcacaaattcgtgaaaggactcaacttcgccgccacctcgataatcaccgctgaagatcccgcagaaaaaatcctttacctgctcaacaaggtcgaagaggcgaagacgttcaaatcgttcactgacttcgatcgtggggtcgaggctgcggtagagctttacgtgaccaacctcaagcacagcgagcacgttgtcagctcctacaagaagaggctctatgaatcgtattaacctgtactaccgtcaaacgcactcgtacgtgcctggctggaagcacctcgactcttacgcctacctcggatacgtgctcgtcaagtcgccggcgcataagctgtacgaagacgacgaggaagtgaagtaccgcggcgtagttcaaatccccagcgagcagcgcaagaaatttcgccgcgtgtggcgccggctcggcacgaaagtgagcttcaacgatttcgtttgcaacgcactggaggactattggaccggtgggtgccagtgcgaacacgactgctgcggctgctttttctactccgcccgcgccaatcgcgtgaaccacaagaagtacaccgtcatcatcaccgggcagcgaaacatatgaccatcgaaatcctgtaccacttcttcgactacctccctgtgtggacggcaccgatatggctgctaccactcctggcgctggcacgataaggaatcaccatgcctgagttcattatcgcgatgttgatctgcacgtcgcctgacgtgtgtataccgttcagggataccactcaacggttcacccaggagcagtgcgaagaagtgcggcaggaaatcacccggcgctacccgcccagcaagttcgcattacgtgtggagtgcgaagttgtggaaccggtggccaagccggcacaaaccagccttatcttctgaggtcactatggaaaacgaaaaacgctgtctcaccaaaaagcgcgacgtgcaactcagtgtcgatgggtacaccctgtccggtggtatcgaactggcacaagacgcgttgtcgatgcgtgtgcagatagcgtccggcaaaaacgaagagtacgaattcgacgtggacactacggtgaccggttggctcgtcgtcaccgtgaagaaacgcgttcccgacgaagcatgggggaaatggagatcgctggcgatttcgccgactgcgctcgtcgagttggccatcggaaccgggctgatcaacgccaacctcgacgttgaagctccgcttcagatcgtagccgtcacaggcgaataatacccttcgggccggaggtcatcatgatcattgcgtcgttcatggacttgtctctggatgtccacgttcgggtcgaatctcggcgggtgaccatgaccctggtcgagtacaagttgtcgtacgtgaagatcaccactggcgagttcatcacgtaccaacctttcttcgaacgttccgcggctgaaacgaaattccgcgagatcgtggaagaccttatcctgaaacagcgaggtac